(19) United States
(12) Patent Application Publication
(10) Pub. No.: US 2016/0228645 A1
(43) Pub. Date: Aug. 11, 2016

(54) NERVE BLOCK INJECTION SYSTEMS AND METHODS

(71) Applicant: Carticept Medical, Inc., Alpharetta, GA (US)

(72) Inventors: Timothy Patrick, Alpharetta, GA (US); Carribeth Ramey, Suwanee, GA (US); Richard Hales, Alpharetta, GA (US); Michael Axelrod, Roswell, GA (US); Thomas Bjurbo, Alpharetta, GA (US)

(21) Appl. No.: 15/017,449

(22) Filed: Feb. 5, 2016

Related U.S. Application Data
(60) Provisional application No. 62/113,224, filed on Feb. 6, 2015.
(60) Provisional application No. 62/168,591, filed on May 29, 2015.

(51) Int. Cl. A61M 5/20; A61M 19/00; A61M 1/00

(52) U.S. Cl. CPC A61M 5/2053; A61M 1/008; A61M 19/00; A61M 2205/3341; A61M 2205/6081

(57) ABSTRACT

According to some embodiments, an injection system configured to deliver at least one nerve block agent, other anesthetic or other fluid to a subject comprises a fluid delivery module configured to receive a fluid container, wherein once secured to the fluid delivery module, the fluid container is configured to engage an actuator of the fluid delivery module, at least one motor coupled to the actuator, wherein the at least one motor is configured to selectively move the actuator to create a positive pressure within the fluid container, a pressure detection assembly in fluid communication with the fluid container, wherein the pressure detection assembly is configured to detect the real-time pressure within the fluid container and the fluid conduit, and a processor configured to regulate at least one aspect of the injection system based on, at least in part, the real-time pressure detected by the pressure detection assembly.

### CROSS-REFERENCE TO RELATED APPLICATIONS

This application claims priority benefit of U.S. Provisional Application Nos. 62/113,224, filed Feb. 6, 2015, and 62/168,591, filed May 29, 2015, both of which are hereby incorporated by reference herein in their entireties.

### BACKGROUND

This application relates generally to injection and/or aspiration devices, systems and methods, and more specifically, to devices, systems and methods of delivering nerve block agents, other anesthetics, other pharmaceuticals or fluids and/or other substances to a subject.

Physicians, clinicians and/or other medical personnel often need to deliver a volume of anesthetic or other medication (e.g., steroid), other fluid and/or other material to or near (or aspirate fluid from) an anatomical location, such as, for example nerve tissue, a joint, an organ and/or the like. Accordingly, a needle can be inserted through a patient's skin and into the targeted location. A syringe or other fluid source that is in fluid communication with the needle can then be used to deliver the desired volume or other dosage of a medicament (e.g., anesthetic), fluid and/or other material to the targeted anatomical location.

For example, physicians, clinicians, and/or other medical personnel often administer anesthetic agents to patients before and/or during surgical and/or other medical procedures. Anesthetics (e.g., nerve block agents) can be delivered to or near particular nerves to cause the temporary loss or reduction of sensation in particular areas of the patient's body. Anesthetics can provide one or more benefits and/or other advantages to patients, such as, for example, decreased post-operative pain, decreased nausea, lower incidence of blood clots, less blood loss, shorter hospital stays after ambulatory surgery, such as orthopedic surgery, a lessened stress response by the body and/or the like. Anesthetics can be particularly appealing to patients undergoing orthopedic procedures, which often involve limbs and are associated with a significant amount of post-operative pain. For example, nerve blocks are often used for procedures used in the repair of certain joints and/or other portions of the anatomy (e.g., shoulders, ACL, knee, elbow, hand procedures wrist, ankle, fingers, toes, etc.).

Anesthetics can also be used for pain management purposes, for example, chronic neck, back, joint pain and/or the like. In some embodiments, an anesthetic injection procedure is accomplished, at least in part, using one or more drugs, other medicaments or fluids (e.g., alcohol or phenol) and/or the like that at least partially destroy nerve tissue. Although anesthetics can provide significant pain relief and offer many advantages and other benefits relating to surgical and/or other medical procedures, it still carries a risk of injury and other complications, such as, for example, inadvertent or unintended nerve damage and adverse effects resulting from a failure to properly locate targeted nerve tissue. Accordingly, various embodiments of improved nerve block delivery systems are disclosed herein.

### SUMMARY

According to some embodiments, an injection system configured to deliver at least one nerve block agent, other anesthetic or other fluid to a subject comprises a fluid delivery module configured to receive a fluid container, wherein once secured to the fluid delivery module, the fluid container is configured to engage an actuator of the fluid delivery module, at least one motor coupled to the actuator, wherein the at least one motor is configured to selectively move the actuator to create a positive pressure within the fluid container, a pressure detection assembly in fluid communication with the fluid container, wherein the pressure detection assembly is configured to detect the real-time pressure within the fluid container and the fluid conduit, and a processor configured to regulate at least one aspect of the injection system based on, at least in part, the real-time pressure detected by the pressure detection assembly.

According to some embodiments, an injection system configured to deliver at least one fluid to a subject comprises a fluid delivery module configured to receive a fluid container, wherein once secured to the fluid delivery module, the fluid container is configured to engage an actuator of the fluid delivery module; at least one motor coupled to the actuator, wherein the at least one motor is configured to selectively move the actuator to create a positive pressure within the fluid container; wherein the fluid container is configured to be placed in fluid communication with a fluid conduit, a distal end of the fluid conduit being configured to receive a needle for placement within a target injection location of the subject. In some embodiments, the system further comprises a pressure detection assembly in fluid communication with the fluid container, wherein the pressure detection assembly is configured to detect the real-time pressure within the fluid container and the fluid conduit; and a processor configured to regulate at least one aspect of the injection system based on, at least in part, the real-time pressure detected by the pressure detection assembly. In some embodiments, wherein the injection system is configured to aspirate a volume of fluid from a subject, wherein the pressure detection assembly is configured to detect a negative pressure within the fluid container and the fluid conduit; and wherein the processor is configured to maintain a generally constant positive pressure within the fluid container and the fluid conduit during delivery of a fluid to a subject to simulate a manually-executed injection procedure to further enhance the safety of an injection procedure.

According to some embodiments, the system is configured to aspirate a volume of fluid from a subject, wherein the pressure detection assembly is configured to detect a negative pressure within the fluid container and the fluid conduit. In some embodiments, the processor is configured to maintain a generally constant positive pressure within the fluid container and the fluid conduit during delivery of a fluid to a subject. In one embodiment, the processor is configured to maintain a variability of the positive pressure within the fluid container and the fluid conduit within a range of 0 to 5 psi. In some embodiments, the processor is configured to maintain the fluid pressure within the container and/or a fluid conduit between 0 to 20 psig during delivery. In some embodiments, the processor is configured to maintain the fluid pressure within the container and/or a fluid conduit between 0 to −5 psig during aspiration.

According to some embodiments, the fluid container comprises a syringe, and wherein the actuator of the fluid delivery module is configured to engage a movable member of the syringe. In some embodiments, the fluid container comprises a standard or non-standard coupling configured to secure to a proximal end of the fluid conduit. In some embodiments, the coupling of the fluid container comprises a luer lock coupling. In one embodiment, wherein the fluid container comprises a syringe, and wherein the actuator of the fluid delivery module is configured to engage a movable member of the syringe.

According to some embodiments, the system comprises a visual indicator relating to a status of a procedure being performed using the system. In some embodiments, such a visual indicator comprises one or more LEDs and/or other lights that are configured to be activated if certain conditions are satisfied. In one embodiment, the visual indicator is activated when delivery of fluids from the system to a subject is commenced and/or ongoing. In some embodiments, the visual indicator is configured to change (e.g., color change, hue change, intensity change, constant indication versus strobe effect, etc.) when a system condition changes (e.g., when the system changes from delivery to aspiration or vice versa, when the pressure (e.g., positive or negative) moves from a safe, satisfactory level to one outside an upper or lower threshold, etc. In some embodiments, the visual indicator comprises at least one light (e.g., LED) configured to change color (e.g., between green, blue, red and/or the like) depending on the status.

According to some embodiments, a distal end of the fluid conduit is configured to receive a stimulation needle. In some embodiments, the fluid container and at least a portion of the pressure detection assembly are included in a unitary assembly that is configured to secure to the fluid delivery module of the injection system. In one embodiment, the at least one membrane of the pressure detection assembly is configured to be in fluid communication with a fluid being transferred between the container and the fluid conduit, the at least one membrane being configured to move upwardly or downwardly relative to the pressure sensor.

According to some embodiments, the fluid delivery module is configured to receive fluid containers of varying sizes and/or shapes. In some embodiments, the processor is configured to automatically terminate an injection procedure when a pressure detected by the pressure detection assembly exceeds a threshold level. In some embodiments, the threshold level comprises a pressure of 20 to 30 psi during fluid delivery and −5 to −7 psi during aspiration.

According to some embodiments, the injection system is configured to be operatively coupled to at least one controller for regulating at least one aspect of the fluid delivery module. In some embodiments, the injection system further comprises at least one controller for regulating at least one aspect of the fluid delivery module. In one embodiment, the at least one controller comprises at least one of a foot pedal and a hand-operated controller. In some embodiments, the hand-operated controller comprises at least one dial, button, switch, dial-pad or touchscreen or other data entry device.

According to some embodiments, the injection system is configured to couple to an imaging device to assist in accurately advancing a needle secured to a distal end of the fluid conduit to a target anatomical location of a subject. In some embodiments, the injection system further comprises an imaging device to assist in accurately advancing a needle secured to a distal end of the fluid conduit to a target anatomical location of a subject. In one embodiment, the imaging device comprises an ultrasound device. In some embodiments, the imaging device is coupled to the fluid delivery module of the injection system using a hardwired or a wireless connection.

According to some embodiments, the injection system is configured to couple to a display, wherein the device is configured to provide data related to an injection procedure being performed using the injection system. In some embodiments, the injection system further comprises a display, wherein the device is configured to provide data related to an injection procedure being performed using the injection system. In one embodiment, the display comprises a display that is integrated with the fluid delivery module. In some embodiments, the display comprises a display that is separate from the fluid delivery module. In certain embodiments, the display is part of a tablet, a smartphone a laptop, another personal computer or another computing device. In one embodiment, the display is coupled to the fluid delivery module using a hardwired or a wireless connection.

According to some embodiments, the fluid contained in the fluid container and configured to be transferred by the fluid delivery module comprises a nerve block agent or another anesthetic. In some embodiments, the processor is configured to aspirate a volume of bodily fluid from a subject before the fluid delivery module is permitted to deliver a fluid within the subject. In some embodiments, the at least one motor comprises a stepper motor or a syringe pump motor.

According to some embodiments, the rate of fluid delivery of fluid from the fluid container through the fluid conduit is not constant in order to maintain a generally constant back pressure during delivery. In some embodiments, the pressure detection assembly comprises a disposable portion and a reusable portion, the disposable portion comprising at least one membrane in fluid communication with fluid being transferred between the fluid container and the fluid conduit. In one embodiment, the processor is configured to maintain a variability of the positive pressure within the fluid container and the fluid conduit within a range of 1 to 4 psi.

According to some embodiments, the fluid container comprises a syringe, and wherein the actuator of the fluid delivery module is configured to engage a movable member of the syringe. In some embodiments, the fluid container comprises a standard or non-standard coupling configured to secure to a proximal end of the fluid conduit. In one embodiment, the coupling of the fluid container comprises a luer lock coupling. In some embodiments, the fluid container comprises a syringe, and wherein the actuator of the fluid delivery module is configured to engage a movable member of the syringe.

According to some embodiments, the injection/aspiration system is portable. In some embodiments, the system comprises at least one battery configured to provide electrical power to the system. In one embodiment, the at least one battery comprises a rechargeable battery (e.g., one or more removable batteries, battery packs, etc.). In some embodiments, the system comprises one or more batteries that are disposable. In some embodiments, the system comprises one or more batteries that can be recharged without removal from the system (e.g., the fluid delivery module). In some embodiments, the system is configured to connect to one or more hardwired power supply systems (e.g., an AC power source, a power unit, etc.).

According to some embodiments, an injection/aspiration system is configured to audibly provide data or other information to a user (e.g., data and/or information regarding real-time fluid pressure, flowrate, volume delivered to and/or removed from a subject, volume remaining in a fluid container, etc.). In some embodiments, the system is configured to audibly provide data or other information to a user using at least one speaker or other audible output device. In one embodiment, the at least one speaker or other audible output device is incorporated into the fluid delivery module of the system. In other embodiments, the at least one speaker or other audible output device is separate from the fluid delivery module of the system.

According to some embodiments, the system is configured to be grasped and held by a user during use. In some embodiments, the system can be grasped and/or otherwise held using a single hand of the user. In some embodiments, the system is configured for convenient transportation.

According to some embodiments, a method of delivering at least one fluid to a subject, the system comprising delivering a fluid from a fluid delivery module of an injection system to a needle located along a distal end of a fluid conduit in fluid communication with the fluid delivery module, wherein the fluid delivery module is configured to receive a fluid container containing the fluid to be delivered to the subject, wherein once secured to the fluid delivery module, the fluid container is configured to engage an actuator of the fluid delivery module, detecting the pressure in real-time of the fluid being transferred by the fluid delivery module to or from the subject via a pressure detection assembly in fluid communication with the fluid container, wherein the pressure detection assembly is configured to detect the real-time pressure within the fluid container and the fluid conduit, and regulating at least one aspect of the injection system using a processor of the system based on, at least in part, the real-time pressure detected by the pressure detection assembly. In some embodiments, the injection system is configured to aspirate a volume of fluid from a subject, wherein the pressure detection assembly is configured to detect a negative pressure within the fluid container and the fluid conduit. In some embodiments, the processor is configured to maintain a generally constant positive pressure within the fluid container and the fluid conduit during delivery of a fluid to a subject to simulate a manually-executed injection procedure to further enhance the safety of an injection procedure.

According to some embodiments, the fluid delivery module comprises at least one motor coupled to the actuator, wherein the at least one motor is configured to selectively move the actuator to create a positive pressure within the fluid container. In one embodiment, the method further comprises aspirating fluid from the subject using the system prior to injecting fluid to the subject to ensure that the needle is properly and safely positioned within the subject. In some embodiments, the pressure detection assembly comprises a disposable portion and a reusable portion, the disposable portion comprising at least one membrane in fluid communication with fluid being transferred between the fluid container and the fluid conduit.

According to some embodiments, the method further comprises displaying at least one data point and/or other information related to the procedure on a display. In some embodiments, the method further comprises imaging the needle as the needle is being advanced within an anatomy of the subject. In some embodiments, the processor is configured to maintain a variability of the positive pressure within the fluid container and the fluid conduit within a range of 1 to 4 psi.

According to some embodiments, the actuator of the fluid delivery module is configured to engage a movable member of the syringe. In some embodiments, the fluid container comprises a standard or non-standard coupling configured to secure to a proximal end of the fluid conduit. In some embodiments, the coupling of the fluid container comprises a luer lock coupling. In some embodiments, the fluid container comprises a syringe, and wherein the actuator of the fluid delivery module is configured to engage a movable member of the syringe.

According to some embodiments, a distal end of the fluid conduit is configured to receive a stimulation needle. In some embodiments, the method further comprises providing at least one stimulation pulse to the needle to ensure a proper and safe response of the subject prior to injecting fluid into the subject. In some embodiments, the fluid container and at least a portion of the pressure detection assembly are included in a unitary assembly that is configured to secure to the fluid delivery module of the injection system.

According to some embodiments, the fluid delivery module is configured to receive fluid containers of varying sizes and/or shapes. In some embodiments, the processor is configured to automatically terminate an injection procedure when a pressure detected by the pressure detection assembly exceeds a threshold level. In some embodiments, the threshold level comprises a pressure of 10 to 30 psi.

According to some embodiments, a processor for regulating at least one aspect of an injection system configured to deliver at least one fluid to a subject comprises a control unit operatively coupled to a fluid delivery module configured to receive a fluid container, wherein once secured to the fluid delivery module, the fluid container is configured to engage an actuator of the fluid delivery module, and an operative connection to at least one motor coupled to the actuator, wherein the at least one motor is configured to selectively move the actuator to create a positive pressure within the fluid container. In some embodiments, the fluid container is configured to be placed in fluid communication with a fluid conduit, a distal end of the fluid conduit being configured to receive a needle for placement within a target injection location of the subject, and an operative connection to a pressure detection assembly in fluid communication with the fluid container, wherein the pressure detection assembly is configured to detect the real-time pressure within the fluid container and the fluid conduit. In certain embodiments, the processor is configured to regulate at least one aspect of the injection system based on, at least in part, the real-time pressure detected by the pressure detection assembly. In some embodiments, the processor is configured to maintain a generally constant positive pressure within the fluid container and the fluid conduit during delivery of a fluid to a subject to simulate a manually-executed injection procedure to further enhance the safety of an injection procedure.

According to some embodiments, the method further comprises audibly providing data or other information to a user (e.g., data and/or information regarding real-time fluid pressure, flowrate, volume delivered to and/or removed from a subject, volume remaining in a fluid container, etc.). In some embodiments, audible data or other information (e.g., alarms, warning, voice, etc.) is provided to a user using at least one speaker or other audible output device. In one embodiment, the at least one speaker or other audible output device is incorporated into the fluid delivery module of the system. In other embodiments, the at least one speaker or other audible output device is separate from the fluid delivery module of the system.

According to some embodiments, the injection/aspiration system is portable. In some embodiments, the system comprises at least one battery configured to provide electrical power to the system. In one embodiment, the at least one battery comprises a rechargeable battery (e.g., one or more removable batteries, battery packs, etc.). In some embodiments, the system comprises one or more batteries that are disposable. In some embodiments, the system comprises one or more batteries that can be recharged without removal from the system (e.g., the fluid delivery module). In some embodiments, the system is configured to connect to one or more hardwired power supply systems (e.g., an AC power source, a power unit, etc.).

According to some embodiments, the system is configured to be grasped and held by a user during use. In some embodiments, the system can be grasped and/or otherwise held using a single hand of the user. In some embodiments, the system is configured for convenient transportation.

### BRIEF DESCRIPTION OF THE DRAWINGS

These and other features, aspects and advantages of the present application are described with reference to drawings of certain embodiments, which are intended to illustrate, but not to limit, the concepts disclosed herein. The attached drawings are provided for the purpose of illustrating concepts of at least some of the embodiments disclosed herein and may not be to scale.

FIG. 1 illustrates a perspective view of an injection system comprising a fluid delivery module configured to selectively deliver fluids to and/or aspirate fluids from a subject according to one embodiment;

FIG. 2 illustrates a perspective view of a fluid delivery module of the injection system of FIG. 1;

FIG. 3 illustrates a bottom view of a fluid container and an adjacent pressure detection assembly configured to be secured to a fluid delivery module of an injection system, according to one embodiment;

FIG. 4 illustrates a side view of the fluid container and the pressure detection assembly of FIG. 3;

FIG. 5 illustrates a partial perspective view of the proximal end of a fluid container where it engages a portion of an actuator of a fluid delivery module of an injection system, according to one embodiment;

FIGS. 6 and 7 illustrate detailed side and top perspective views, respectively, of the pressure detection assembly of FIG. 4;

FIG. 8 illustrates various embodiments of screenshots provided by a display operatively coupled and/or incorporated into an injection system, according to some embodiments;

FIG. 9 illustrates one embodiment of a foot pedal configured to be used as a controller for regulating at least one aspect of an injection system;

FIG. 10 illustrates another embodiment of an injection system comprising a fluid delivery module configured to selectively deliver fluids to and/or aspirate fluids from a subject;

FIG. 11 illustrates a different embodiment of an injection system comprising a fluid delivery module configured to selectively deliver fluids to and/or aspirate fluids from a subject;

FIG. 12 illustrates another embodiment of an injection system comprising a fluid delivery module configured to selectively deliver fluids to and/or aspirate fluids from a subject;

FIG. 13 illustrates the injection system of FIG. 12 with a close-up view of the pressure detection assembly separated; and

FIG. 14 illustrates one embodiment of a foot pedal configured for use with an injection system.

### DETAILED DESCRIPTION

In some embodiments, regional, peripheral or local anesthesia (e.g., a nerve block) involves the application of one or more local anesthetic agents or anesthetics to or near nerves (e.g., individual nerves, nerve bundles, other nerve tissue, etc.). As used herein, the term anesthetic is a broad term and includes, without limitation, any pharmaceutical, medicament, formulation and/or any other fluid, substance or material that causes anesthesia or a loss (e.g., reversible or permanent loss) of sensation. Anesthetics include, but are not limited to, local or regional anesthetics, analgesics and/or other painkillers or pain relieving materials, medicaments, materials and/or substances (e.g., alcohol, phenol, etc.) that at least partially destroy nerve tissue and/or the like. The terms anesthetic, anesthetic agent, nerve block and the like are used interchangeably herein. Anesthetics can include pharmaceuticals and/or formulations or combinations thereof. Further, anesthetics can include one or more substances that are also configured to provide at least some benefit or effect on nerves and related tissues, including cooling or cryogenic materials, other numbing or related substances and/or the like.

Several embodiments disclosed herein are particularly advantageous because they include one, several or all of the following benefits or advantages: providing real time positive pressure and negative pressure (e.g., vacuum) detection and determination of fluid being transferred to and/or from a subject, providing detection and determination of actual pressure (e.g., positive and/or negative) of fluid being transferred (e.g.,. fluid passing adjacent a pressure detection module) without the need to extrapolate or predict such pressure, providing a compact, portable unit that has a reduced footprint; providing an injection/aspiration system that is configured for accurate delivery or aspiration of fluids to and/or from a subject; providing a system that conveniently visually (e.g., using LED or other backlight features) and/or audibly provides status information related to one or more aspects of a procedure; and/or the like.

In some embodiments, anesthetics (e.g., nerve blocks) are used for surgical procedures, pain management purposes and/or any other clinical purpose or reason. For example, pain caused by inflammation of joints (e.g., facet joints, other portions of the spine, other joints, etc.) and/or other portions of the anatomy can be at least partially alleviated through the injection of one or more anesthetics to or near nerves associated with such pain. In some embodiments, injections of local anesthetics into a corresponding joint capsule or surrounding tissue can help relieve such inflammation and pain. By way of example, certain types of anesthetics that are used for various indications include, but are not limited to, interscalene plexus block, supraclavicular plexus block, infraclavicular plexus block, axillary plexus block, median nerve block, radial nerve block, ulnar nerve block, femoral nerve block, popliteal nerve block, tibial block, deep peroneal block, saphenous nerve block, sural nerve block, paravertebral block, Transversus Abdominis Plane (TAP) block, sciatic block and/or the like. Accordingly, anesthetics can be selectively delivered to any portion of the anatomy including, but not limited to: the shoulder, upper arm, elbow, forearm, wrist, hand, thigh, femur, knee, jaw, foot, toes, fingers, spine, neck, internal organs, and/or the like.

Anesthetics can be generally classified into two groups—the ester group and the amide group. The difference in chemical structure of the two groups affects the pathway by which the drugs are metabolized, as well as the potential for allergic reaction. Ester anesthetics are metabolized by hydrolysis, while amides are metabolized by microsomal enzymes located in the liver. By way of example, ester anesthetics that can be delivered using one or more of the injection system embodiments disclosed herein include, but are not limited to, cocaine, procaine, chloroprocaine, tetracaine, and/or the like. Further, amide anesthetics that can be delivered using one or more of the injection system embodiments disclosed herein include, but are not limited to, lidocaine, mepivacaine, bupivacaine, etidocaine, prilocaine, and/or the like. However, any other type of local or regional anesthetic and/or other type of anesthetic can be delivered using the various injection system embodiments, disclosed herein. One or more anesthetics can be delivered using one or more of the injection system embodiments disclosed herein either alone or in combination with one or more other fluids, medicaments and/or other substances or materials. As discussed in greater detail herein, such anesthetics and/or other substances can be delivered by the injection system either sequentially or concurrently, as desired or required. In addition, in any of the embodiments of an injection system disclosed herein, one or more fluids (e.g., anesthetics, steroids, other anti-inflammatory, etc.) can be delivered to a targeted portion or region of the anatomy either alone or in combination with another treatment step or procedure, such as, for example, energy delivery. For example, in some embodiments, a needle secured to the injection system can be configured to provide one or more forms of energy, such as, radiofrequency (RF), ultrasound, microwave, laser and/or the like. Such energy forms can be used to modulate, ablate and/or otherwise affect native tissue of the patient. For instance, such energy delivery can be configured to ablate tissue (e.g., create lesions), stimulate, modulate and/or ablate nerve tissue (e.g., denervation, neural modulation, etc.), enhance the efficiency and therapeutic effect accompanying the delivery of medicaments and/or other materials into the patient and/or the like.

One or more anesthetics can be delivered alone or in combination with another fluid or substance, for example, epinephrine. In some embodiments, anesthetic agents can be delivered for a duration ranging, for example, from about 15 minutes to about 240 minutes. According to some embodiments, for example, the duration of delivery can be about 15 to about 30 minutes, about 30 to about 60 minutes, about 45 minutes, about 30 to about 120 minutes, about 120 to about 240 minutes, about 200 minutes, less than about 45 minutes, more than about 240 minutes, or any duration or range of durations between these example durations and ranges. Such anesthetic agents in combination with, for example, epinephrine and/or any other substance, can be delivered for a duration ranging, for example, from about 30 minutes to about 480 minutes. For example, the duration of delivery can be about 30 to about 90 minutes, about 30 to about 120 minutes, about 60 to about 400 minutes, about 240 to about 280 minutes, about 240 to about 360 minutes, about 240 to about 480 minutes, less than 30 minutes, more than 480 minutes, or any duration or range of durations between these example durations and ranges. In some embodiments, the duration provided herein are configured for continuous infusion applications. Alternatively, the delivery duration can be selected for single shot blocks. For example, in some embodiments, the actual duration of delivery is less than 5 minutes total (e.g., 0-30 seconds, 30-60 seconds, 1-2 minutes, 2-3 minutes, 3-4 minutes, 4-5 minutes, time values between the foregoing ranges, etc.). In other embodiments, for single shot blocks, delivery duration can be greater than 5 minutes (e.g., 5-10, 10-20 minutes, more than 20 minutes, etc.). Irrespective of the exact delivery duration, in some embodiments, the entirety of a procedure performed using the injection/aspiration device may exceed the delivery duration, as desired or required. For example, in some embodiments, last more than 5 minutes (e.g., 5-10, 10-20, 20-30 minutes, more than 30 minutes, etc.).

In some embodiments, anesthetic agents and/or other medicaments or materials are delivered up to a maximum dose in the range of, for example, about 1.5 mg/kg to about 11.5 mg/kg. For example, a maximum dose of an anesthetic agent can be about 1.5 mg/kg, 2.5 mg/kg, 2.8 mg/kg, 4.2 mg/kg, 4.5 mg/kg, 5.7 mg/kg, 7.1 mg/kg, 11.4 mg/kg, less than 1.5 mg/kg, more than 11.5 mg/kg, or any other dose between these example doses. In some applications, such anesthetic agents in combination with, for example, epinephrine, can be delivered up to a maximum dose in the range of, for example, about 3.2 mg/kg to about 14.2 mg/kg. For example, a maximum dose of an anesthetic agent in combination with, for example, epinephrine, can be about 3.2 mg/kg, 5.7 mg/kg, 7.0 mg/kg, 8.5 mg/kg, 14.2 mg/kg, less than 3.2 mg/kg, more than 14.2 mg/kg, or any other dose between these example doses.

Anesthetic injection or delivery procedures can be time-consuming, and often involve two, three, or more clinicians for a single procedure. Inefficiencies associated with certain tasks, such as, for example, manual syringe preparation and drug and patient data recording, can contribute to the time and labor required for such procedures. The devices, systems, and methods discussed herein can provide more accurate, precise, and controlled delivery of local anesthetic agents (e.g., nerve block agents), controlled aspiration, as well as automated record keeping, which can enhance quality control and allow for more efficient nerve block procedures. In some embodiments, such devices, systems, and methods can advantageously allow for anesthetic delivery procedures to be performed with fewer personnel and resources, such as, for example, only one or two clinicians. Thus, the demand and associated cost of additional clinicians (e.g., anesthesiologists, other physicians, nurses, other healthcare professionals, etc.) can be advantageously reduced.

Although the devices, systems, and methods described herein are often discussed in the context of nerve block or anesthesia injection procedures, which may have applicability to intra-articular or other anatomical injection procedures, the devices, systems, and methods can also be adapted for use for any other indication and/or for any other purpose. For example, any of the injection systems disclosed herein or variants thereof can be used to delivery cancer or other relatively potent medications to a particular portion of the anatomy (e.g., to or within a specific diseased organ, internal blood vessel, etc.). In other embodiments, the injection system can be used to deliver contrast agent to a particular organ and/or other portion of the anatomy. In some embodiments, the injection system is used for biopsies or diagnostic procedures where a volume or mass of a native bodily fluid and/or tissue (e.g., from within an organ, cyst, cavity, etc.) is extracted (e.g., using aspiration features of the injection system). In such biopsy or diagnostic procedures, the system can be used to selectively deliver one or more medicaments and/or other fluids (e.g., anesthetics, contrasting agents, etc.) to the targeted anatomical location.

According to some embodiments, the injection system is used for epidural analgesia or spinal anesthesia procedures. For example, in such embodiments, epidural analgesia is the anesthetic that is delivered, either alone or in combination with one or more other medicaments or fluids, to a patient's epidural space. In any of the embodiments disclosed herein, the injection system can be used to deliver one, two or more medicaments (e.g., nerve block agents, anesthetics, etc.) and/or other materials to a targeted anatomical location through a needle, catheter, another body-inserted tube or lumen and/or combinations thereof, as desired or required. For example, in epidural or other spinal injections, the injection system can be used to deliver one or more medicaments (e.g., nerve block agents, anesthetics, etc.) and/or any other fluid or substance either through a percutaneous needle secured to a distal end of the system or through a catheter positioned within the patient. Regardless of the exact indication or manner of delivery, an injection of one or more anesthetics can advantageously block, at least partially, the transmission of signals through nerves to offer one or more advantages and/or benefits to the patient (e.g., temporary or long-term pain relief or pain reduction).

The discussion and the figures illustrated and referenced herein describe various embodiments of an injection and aspiration device and system, as well as methods related thereto. A number of these embodiments of injection/aspiration systems, devices and methods are particularly well suited to transfer a volume of one or more fluids and/or other materials (e.g., nerve block agents, anesthetics, etc.) to or near (and/or from) a location of the human anatomy such as a nerve (e.g., nerves, nerve bundle, nerve tissue, etc.). Such devices, systems and methods are well-suited for inducing and maintaining anesthesia during surgical and/or other medical procedures. However, the various devices, systems, methods and other features of the embodiments disclosed herein may be utilized or applied to other types of apparatuses, devices, systems, procedures and/or methods, regardless of whether they are medically-related or not.

As discussed in greater detail herein, this application discloses devices, systems and methods of locating target nerve tissue or region or other anatomical location and delivering and/or withdrawing fluids and/or other materials (e.g., nerve block agents, anesthetics, other medications, pharmaceutical compositions, drugs, cells (e.g., stem cells and other biologics), liquid and non-liquid fluids and flowable materials, nanoparticles, cement, microbeads, etc.) thereto and/or therefrom. According to some embodiments, the devices, systems and methods disclosed herein facilitate the delivery and/or aspiration of fluids and/or other materials to and/or from a nerve or other anatomical location by advantageously using a single needle penetration. Other target anatomical locations include, but are not limited to, intra articular spaces (e.g., joints), soft tissue, dermal/subdermal tissue, organs and/or the like. Several embodiments of the inventions disclosed herein are particularly advantageous for the anesthesiology and dermatological (aesthetics) fields. The fluids and/or other materials can vary in type (e.g., formulation), strength (e.g., concentration) and/or in any other manner. The delivery of anesthetic agents, other fluids and/or other materials to or near nerves and/or other anatomical locations using the embodiments disclosed herein can help decrease the risk of complications during surgical procedures. Such systems, devices and methods can be especially useful for the treatment of smaller target areas. In addition, such devices and methods can simplify the execution of related procedures by physicians and other medical personnel. Accurately locating a target nerve traditionally has been a relatively difficult task. According to some embodiments, the devices, systems and methods disclosed herein help a clinician or other user to locate targeted nerves or other anatomical spaces for the subsequent accurate delivery of fluids and/or other materials thereto.

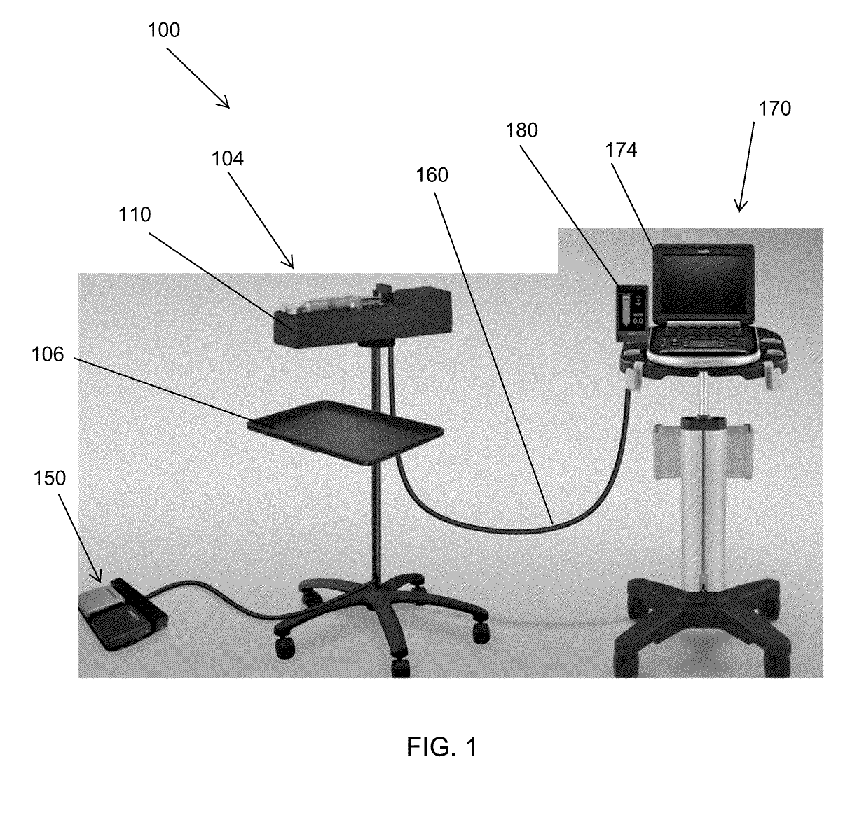

One embodiment of an injection system 100 for delivering one or more nerve block agents, other anesthetics and/or other fluids to a subject is illustrated in FIG. 1. As shown, the system 100 can include one or more carts 104, 170 and/or other movable or stationary portions. For example, in the illustrated embodiment, the system 100 comprises a first cart 104 that is configured to receive a fluid delivery module 110. As shown, the cart 104 can further include one or more trays, shelves and/or other receiving areas or portions 106. In some embodiments, such portions 106 can be designed to facilitate a surgeon or other user in executing a particular procedure.

With continued reference to FIG. 1, the system 100 can include one or more controllers 150 and/or other features that enable a physician or other practitioner or user to advantageously regulate one or more aspects of a procedure. In the depicted embodiment, the controller 150 comprises a foot pedal device (e.g., a single pedal, a dual pedal, a pedal configured to be rocked or moved in a manner that permits a user to select different modes (e.g., aspiration or delivery), etc.); however, in other configurations, one or more other types of controllers can be used, either in lieu of or in addition to the foot pedal. For example, a controller can include, without limitation, one or more buttons (e.g., multi-mode buttons, multi-depth buttons, etc.), rheostats, dials, knobs, switches, rollerballs, rollerwheels, combinations thereof and/or the like. Such controllers can be attached (e.g., removably, permanently, etc.) to one or more other components of the system 100 (e.g., the fluid delivery module 110, an imaging device, etc.), as desired or required. Alternatively, such controllers can be separate from other system components, such as, for example, the foot pedal 150 illustrated in FIG. 1.

According to some embodiments, the fluid delivery module 110 of the injection system 100 can be operatively coupled to a display 180 and/or an imaging device 174. The display 180 and/or the imaging device 174 can be physically attached to or can be separate from the fluid delivery module 110, as desired or required. For example, in the illustrated embodiment, both the display 180 and the imaging device 174 are physically separate from the fluid delivery module 110, and positioned on a separate cart 170. However, in other arrangements, one or both of the display 180 and the imaging device 174 can be integrated with (e.g., can have a unitary or monolithic structure in relation to) at least a portion of the fluid delivery module 110 (e.g., a housing of the module 110). In other embodiments, the display 180 and/or the imaging device 174 can be configured to be removably or permanently secured to at least a portion of the fluid delivery module 110.

As discussed with reference to other embodiments herein, the imaging device 174 that is either incorporated into the injection system or operatively coupled to the injection system 100 can comprise an ultrasound device that is configured to detect and track the location of a needle or other component or member (positioned along a distal end of a handpiece or other conduit that is placed in fluid communication with the fluid delivery module of the system) as a surgeon or other user of the system advances the needle or other distal component or member within the anatomy of a subject. However, in other embodiments, any other type of imaging device or system can be used, such as, for example, fluoroscopy, CT, MRI and/or the like, either in lieu of or in addition to ultrasound. Regardless of their exact configuration and design, such systems can be separate (e.g., off-the-shelf) units that can be configured to integrate and/or otherwise work with any of the injection system embodiments disclosed herein or variations thereof.

In some embodiments, the display 180 is integrated into the design of the fluid delivery module 110. However, in other embodiments, the display 180 is a separate device, such as depicted in FIG. 1 herein. In some embodiments, the display 180 can be part of a separate computing device, such as, for example, a laptop computer, another type of personal computer, a larger network system, a smartphone, a tablet and/or the like. Thus, a user can advantageously display data, images and/or other information related to the system and/or the procedure being performed using a readily-available and/or custom device. This can help reduce overall cost of the system, simplify the overall design of a system, facilitate integration with existing systems and components and/or provide one or more additional advantages or benefits to users. In some embodiments, special software (e.g., applications) can be used to integrate a separate device comprising a display with the injection system, as needed or required.

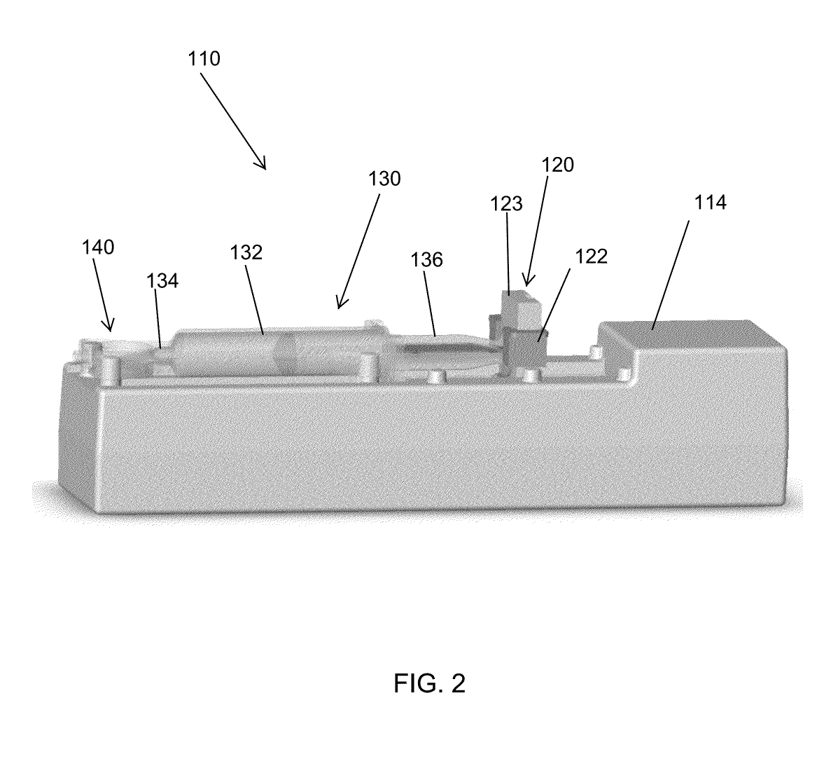

FIG. 2 illustrates one embodiment of a fluid delivery module 110 configured to selectively deliver one or more fluids to a subject. As discussed herein with reference to other injection system arrangements, the fluid delivery module 110 can include one or more motors and/or other electromechanical components to facilitate the execution of a procedure. In addition, the fluid delivery module 110 can be configured to securely receive one or more containers 130 containing anesthetics (e.g., nerve block agents) and/or any other fluids or materials. For example, as illustrated in FIG. 2, an upper surface of the fluid delivery module 110 can be designed and/or otherwise configured to receive a syringe 130. For example, the fluid delivery module 110 can include one or more syringe receiving slips, slots and/or other features or members. In some embodiments, the syringe 130 comprises a pre-filled syringe, as provided to the user by a drug manufacturer or supplier. Alternatively, however, such syringes or other fluid containers (containing, e.g., nerve block or other anesthetic) can be filled (or re-filled) on site by the user of the system. The syringes or other containers 130 configured to secure to the fluid delivery module 110 can be standard or non-standard, as desired or required for a particular application or use.

With continued reference to FIG. 2, the fluid delivery module 110 can comprise a housing or other outer portion 114 that encloses (e.g., at least partially) one or more internal components, such as, for example, motors, actuators, processors, memory units, controllers, wires or other electrical connections and components and/or the like. In embodiments where the fluid delivery module 110 is configured to receive a syringe or similar container 130, a plunger or other movable member 136 of the container 130 can include one or more features or portions that are sized, shaped and/or otherwise adapted to engage an actuator assembly 120 of the motor (not shown in FIG. 2). The actuator assembly 120 can include an actuator 122 that is sized, shaped, positioned and/or otherwise configured to engage a plunger or other portion of the syringe 130 or other container loaded on a corresponding receiving area of the injection system. For example, in the depicted embodiment, a proximal end of the plunger 137 is designed to be placed within a corresponding recess or opening of an actuator 122. As discussed with reference to other arrangements herein, the actuator can be integrated with and/or otherwise coupled (e.g., directly or indirectly) to one or more motors of the module 110. In some embodiments, the motor of the fluid delivery module 110 comprises a stepper motor or another type of syringe motor that is capable of delivering and/or aspirating fluids against the expected forces during an injection/aspiration procedure. In some embodiments, as shown in FIG. 5, a locking device 123 can be used to provide additional security that the plunger and/or other movable member 136 of the container remains properly positioned relative to the actuator 122 during use.

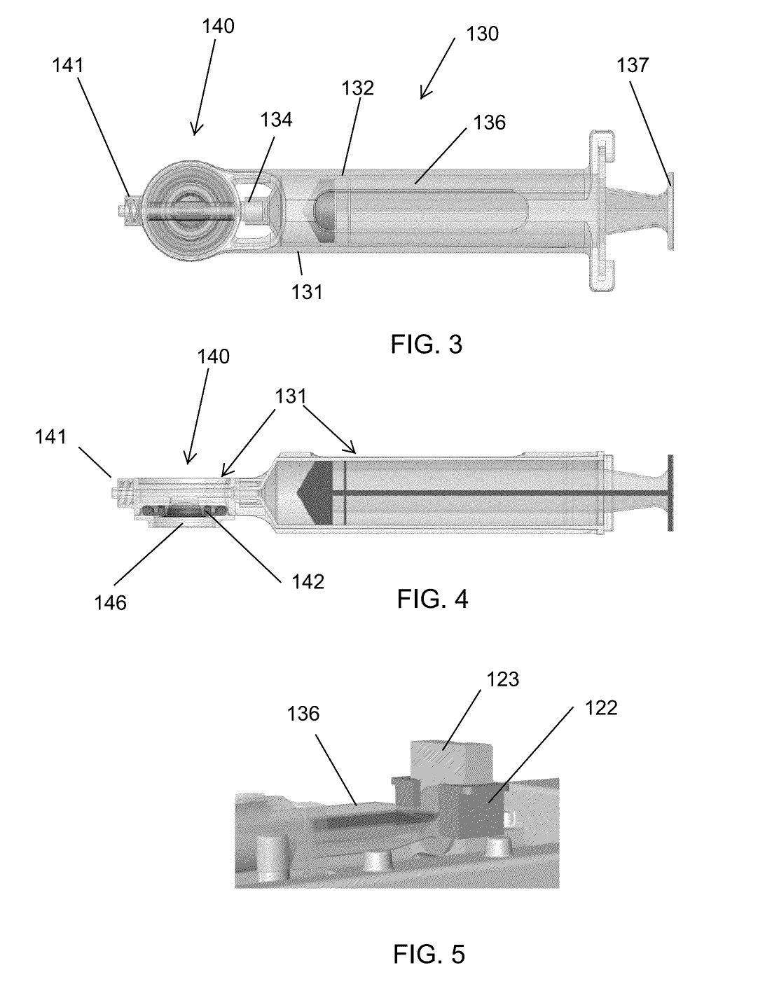

According to some embodiments, as illustrated for example in FIGS. 3 to 7, a pressure detection assembly 140 can be positioned between the fluid container 130 and the conduit (e.g., tubing, handpiece, etc.—not shown) that transfers fluid from and/or to the subject. As shown, the pressure detection assembly 140 can be positioned immediately downstream of the fluid container 130 (e.g., syringe). In some embodiments, as illustrated in FIGS. 3 to 7, the container (e.g., syringe) 130 is integrated, at least in part, into a single housing or structure 131 with the pressure detection assembly 140. However, in other embodiments, the pressure detection assembly 140 is positioned at a different location relative to the container 130 and/or the fluid delivery module 110. For instance, the pressure detection assembly 140 can be located along the fluid conduit (not shown) that places the syringe or other container 130 secured to the fluid delivery module 110 in fluid communication with a conduit and needle (not shown).

As discussed in greater detail herein, the pressure detection assembly 140 can be configured to accurately detect a fluid pressure (e.g., positive or negative) being transferred by the fluid delivery module 110 of the system 100. In some embodiments, a portion of the pressure detection assembly 140 is configured to come in contact (e.g., directly or indirectly) with the fluid being transferred through or near the pressure detection assembly. Such components or portions of the assembly 140 can be configured to be disposable or replaceable between procedures. Further, in some embodiments, the pressure detection assembly 140 comprises one or more reusable components or portions, such as, for example, a pressure sensor (not shown), associated electric connections, electronic components and/or the like. In such configurations, the reusable components or portions (e.g., sensor) can be integrated directly into the fluid delivery module 110 or any other portion or component of the injection system 100.

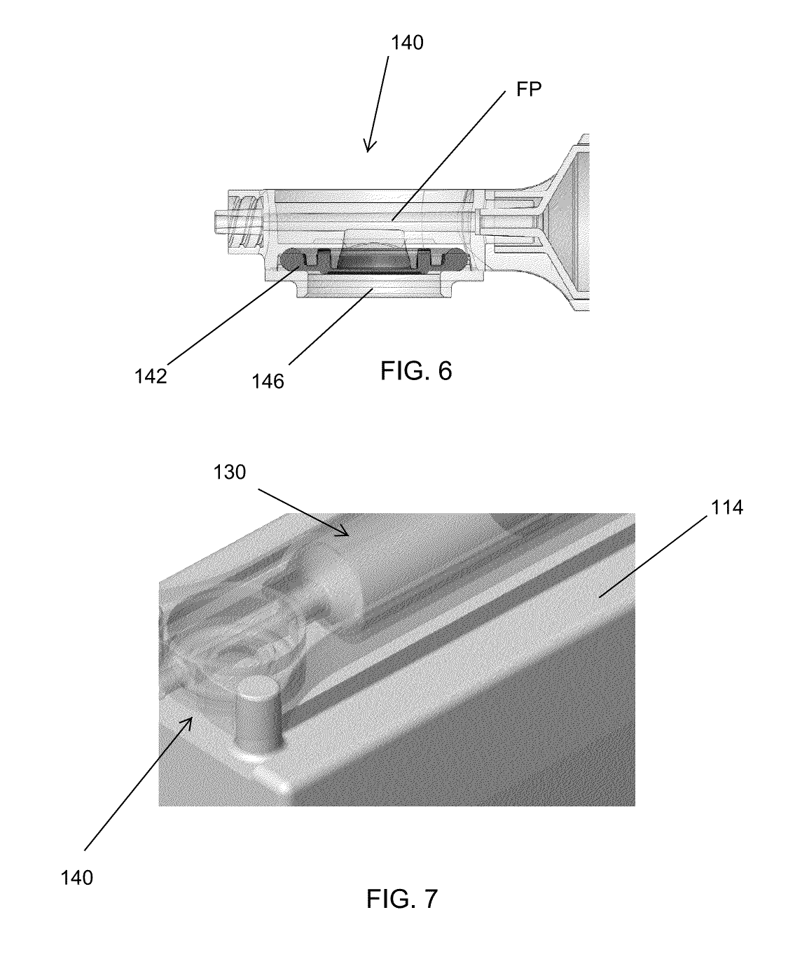

As shown in FIG. 6, the pressure detection assembly 140 comprises a movable member 142 that is in fluid communication with the fluid path FP extending through the assembly 140. In some embodiments, movable member 142 comprises a membrane (e.g., disc, diaphragm, other flexible member, etc.) that is configured to move up or down and/or otherwise respond to the pressure created by the adjacent fluid, depending on the fluid pressure within the adjacent fluid path FP. In the depicted embodiment, only an upper portion of the movable member 142 extends into the fluid path FP and makes contact with the fluid passing therethrough. However, in other embodiments, the portion or extent of the movable member 142 that actually contacts the fluid being transferred using the fluid delivery module 110 can be greater or less than illustrated in FIG. 6.

Depending on the pressure of the fluid passing through the fluid path FP of the pressure detection assembly 140, the movable member 142 will be deflected or moved (e.g., either upwardly or downwardly, depending on if the fluid pressure is positive or negative). Thus, if the movable member 142 is operatively coupled to a pressure sensor (not shown), the pressure of the fluid passing through the pressure detection assembly 140 can be accurately determined. In some embodiments, the use of such movable members 142 that are at least in part in direct dynamic communication with the fluid being transferred using the injection system 100 can provide for more accurate, real-time pressure readings. This can advantageously improve the execution of an injection (with or without aspiration) procedure, allowing the physician or other practitioner to more accurately and safely delivery fluids to a subject (and/or aspirate fluids from the subject).

With continued reference to FIG. 6, as noted above, the pressure detection assembly 140 can allow a pressure sensor and/or other component to be adjacent (e.g., immediately or directly, indirectly, etc.) the membrane or other movable member 142 that is configured to move upwardly or downwardly in response to fluid pressures within the fluid path FP. For example, in FIG. 6, a pressure sensor (e.g., transducer, strain gauge, load cell, etc.) can be incorporated (e.g., permanently or removably) into a portion of the fluid delivery module 110 (e.g., along a portion of the outer housing, a recess within the housing, etc.). Pressure sensors can include, without limitation, mechanical pressure sensors or any other type of pressure sensors (e.g., piezoelectric pressure sensors, electromechanical pressure sensors, capacitive pressure sensors, electromagnetic pressure sensors, optical pressure sensors and/or the like). Thus, in some embodiments, a pressure sensor or a component operatively coupled to it is configured to be placed, at least partially, within a space 146 adjacent the movable member of the pressure detection assembly 140.

As illustrated in FIG. 6, the membrane or other movable member 142 of the pressure detection assembly 140 can include a design and/or configuration to secure the member 142 within the corresponding structure of the assembly 140. For example, as shown, the movable member 142 includes flanged or flared outer features that are shaped, sized and/or other adapted to fit within and secure to corresponding recesses or features of adjacent portions of the pressure detection assembly 140. Thus, in some embodiments, the movable member 142 is securely retained within the assembly 140 using one or more positive engagement or locking features. However, in other arrangements, the membrane or other movable member 142 is configured to be secured within the pressure detection assembly 140 using one or more other devices or methods, such as, for example, adhesives, mechanical connections, friction fit or press fit connections and/or the like, as desired or required. As noted above, in some embodiments, the syringe (or other fluid container) and at least a portion of the pressure detection assembly 140 can be provided as a single unit or structure 131. In some embodiments, such a single unit or structure 131 can be configured to be removed and replaced between procedures.

As illustrated in FIGS. 3, 4, 6 and 7, an output or outlet (or other distal portion) of the container (e.g., syringe) 130 can be connected to and placed in fluid communication with the pressure detection assembly 140 using one or more connection devices, features and/or methods. For example, the two portions can be placed in fluid communication with one another using standard or non-standard connectors or couplings, such as, for example, luer lock couplings, friction fit or press fit connectors and/or other like. In other embodiments, where the container 130 is physically separated from the pressure detection assembly, the two components 130, 140 can be placed in fluid communication with one another using one or more conduits (e.g. tubing, other fluid connectors or couplings, etc.).

According to some embodiments, a processor of the injection system (e.g., which may be contained, completely or partially, within the fluid delivery module, which may be separate of the fluid delivery module, etc.) is configured to operate the fluid delivery module (e.g., the motor and linear actuators contained therein) to execute an injection procedure in a safe, accurate, predictable and desired manner. For example, according to some embodiments, the processor is operated according to one or more algorithms or operational schemes that are configured to perform or otherwise carry out an injection procedure in a manner than reduces the likelihood of harm to the subject and/or simulates how a practitioner would otherwise manually inject a nerve block agents or other anesthetic into a subject's target anatomical location.

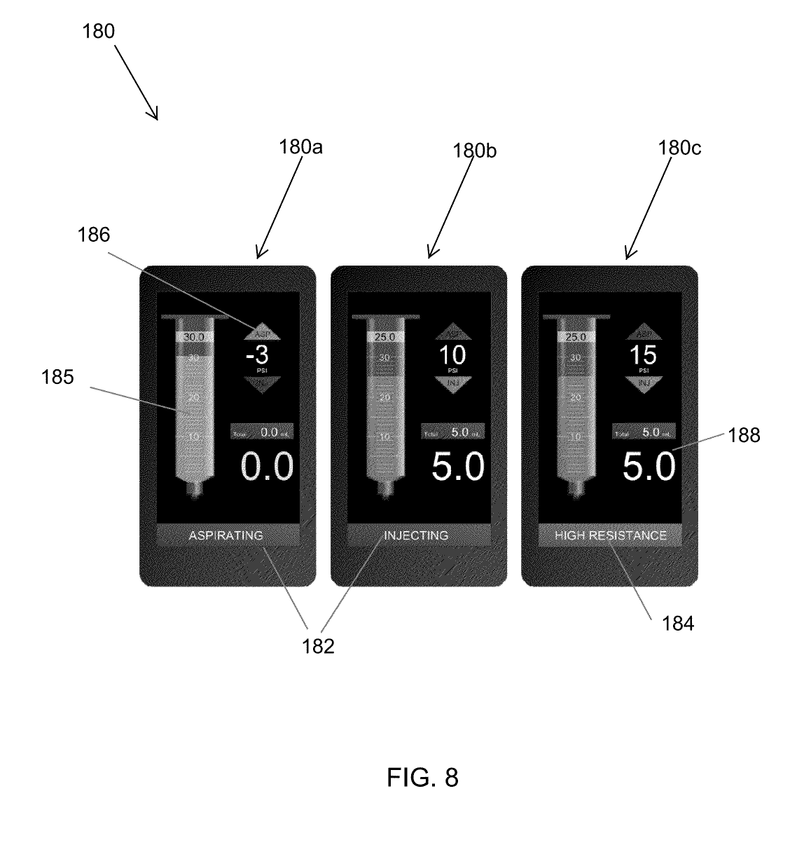

As illustrated in the embodiments of FIG. 8, data and/or other information regarding an injection procedure can be displayed on one or more displays or other output devices or components that are incorporated or otherwise operatively coupled to the injection system 100. As noted herein, the display 180 can be included within one or more components of the injection system 100, such as, for example, the fluid delivery module 110. However, in other configurations, a display from a separate computing device (e.g., tablet, smart phone, laptop, other computing device, etc.) can be adapted to provide information to the user.

Regardless of the type of display and the exact manner in which such a display 180 is integrated into an injection system 100, the display can be configured to provide data or other information about an injection procedure to facilitate the execution of such a procedure. For example, as illustrated in FIG. 8, the display can provide information 185 (e.g., in the form of data, a visual or graphical representation, etc.) regarding the volume of a fluid (e.g., nerve block agent, other anesthetic, other fluid, etc.) that has been delivered and/or that is remaining within the fluid container 130 loaded onto a fluid delivery module 110 of the system 100.

With continued reference to FIG. 8, the display 180 can provide the pressure data 186 (e.g., real-time positive or negative pressure, depending on whether fluid is being delivered to or aspirated from the subject). Relatedly, the display can indicate whether the system is in the process of injecting or aspirating fluids 182, 186. Additional data and/or other information can also be provided, as desired or required, such as, for example, alarms or other alerts 184 that the pressure (e.g., positive or negative) exceeds a pre-set threshold (e.g., maximum or negative) value.

According to some embodiments, the system comprises voice feedback configured to provide a warning, an alert, data and/or other information to a user. For example, in some embodiments, when the real-time pressure detected by the system reaches or exceeds a particular threshold level (e.g., which in some embodiments, can be adjusted by the user), the system is configured to audibly provide feedback to the user. For example, in some embodiments, the system is configured to warn or otherwise alert the user that the fluid pressure (e.g., positive pressure, suction or vacuum) has reached or surpassed the threshold level via an alarm or other sound (e.g., beep). In other embodiments, the system is configured to provide information to the user via a computer-generated voice feature. For example, once the pressure has reached or exceeded the threshold, the system is configured to activate a voice (e.g., replicating a human voice) that provides certain information to the user (e.g., “warning, the fluid pressure is at or above the threshold level”). In other embodiments, the system is configured to warn or otherwise alert the user when a particular threshold (e.g., upper or lower pressure) is close to being attained. As discussed in greater detail herein, one or more components of the system (e.g., the fluid delivery module) can be configured to provide feedback regarding the status of a procedure being executed using the system using one or more visual features. For example, the fluid delivery module can be configured to change colors (e.g., using one or more LEDs) depending on whether the system is presently delivering fluid to a subject or aspirating fluid from a subject. In some embodiments, for example, the fluid delivery module (and/or any other component of the system) can be configured to provide a green light when fluid delivery to a subject is occurring and a blue light when aspiration is occurring. Any other light or visual indication can be used in lieu of green and blue lights, as desired or required. Further, such a visual configuration can be adapted to alert the user when the delivery and/or aspiration pressure exceeds a particular threshold (e.g., which may be fixed or adjusted by the user). By way of example, if the delivery or aspiration pressure is exceeded, the fluid delivery module can be configured to emit a red (or some other light color or visual alert, e.g., strobe effect) to alert the user of such event.

In other embodiments, an audible warning or other information (e.g., alarm, voice, etc.) is provided to the user for other parameters, either in addition to or in lieu of pressure. For example, in some embodiments, information relating to flowrate, volume delivered, volume remaining and/or the like can be audibly provided to a user by the system. In some embodiments, the system is configured to provide data and/or other information related to an injection or an aspiration procedure according to a particular frequency. Such a frequency can be variable, fixed, predetermined, adjustable and/or the like. In one embodiment, a user can select (e.g., customize) the type of data and/or other information that will be provided audibly, the frequency at which such data and/or information will be provided audibly and/or the like. For example, a user can choose to have real-time pressure and volume delivered to a subject (and/or volume of fluid remaining to be delivered) audibly provided according to a pre-set (e.g., fixed) or a desired frequency. For instance, a user can choose to have selected data and/or information provided according to his or her desired frequency (e.g., every 5 to 10, 10 to 20, 20 to 30 seconds, 30 seconds to 1 minute, 1 to 2, 2 to 3 minutes, time intervals between the foregoing, etc.). The system's ability to audibly provide data and/or other information during the execution of an injection and/or aspiration procedure can be in lieu or in addition to data and/or other information provided via one or more displays and/or other output devices (e.g., displays, other output device and/or the like that are integrated with or separate of the system).

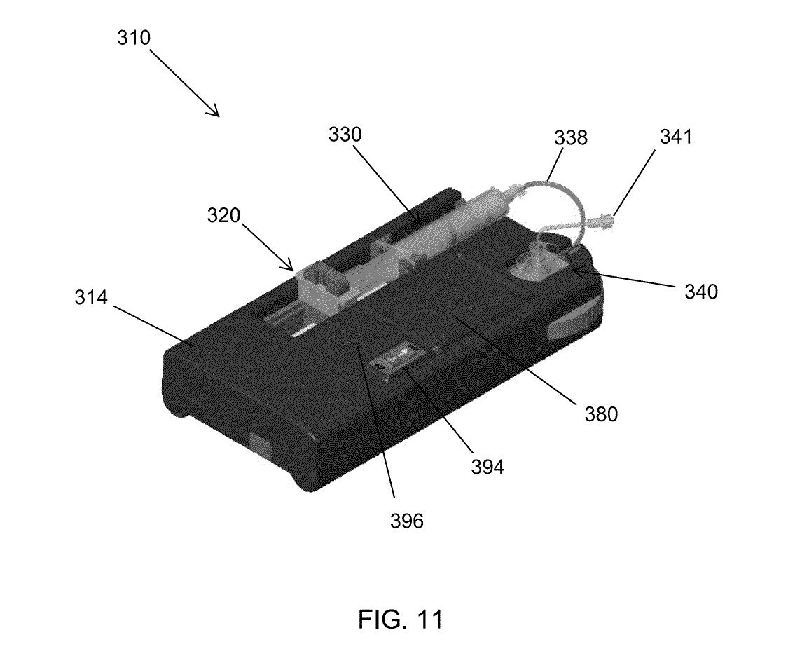

Accordingly, any of the devices and systems disclosed herein can comprise one or more speakers or other audible output devices. For example, as illustrated in the injection system 310 depicted in FIG. 11, one or more speakers 396 can be provided along or near the housing 314 of the system. As discussed, the system can comprise the necessary processor and/or other components to deliver the desired or required alerts and/or other audible output to the user. In some arrangements, one or more speakers can be external to the injection device or system. For instance, the desired audible output can be provide via one or more stand-alone speakers or through one or more intermediate devices (e.g., a computer, a network and/or the like).

In some embodiments, the system can be configured to provide haptic or other motion-based feedback during use. For example, the fluid delivery module, a handpiece that operatively couples to the injection/aspiration system and/or any other component of the system can be configured to vibrate or other alert the user that an event has occurred or is about to occur. By way of example, in some embodiments, the fluid delivery module and/or another component of the system can include one or more vibrating or otherwise movable members that cause the system to move when the upper (e.g., positive) and/or lower (e.g., negative) threshold fluid pressure has been reached or is near. Such haptic or similar alerts can be provided either in lieu or in addition to any audible feedback that is provided by a system.

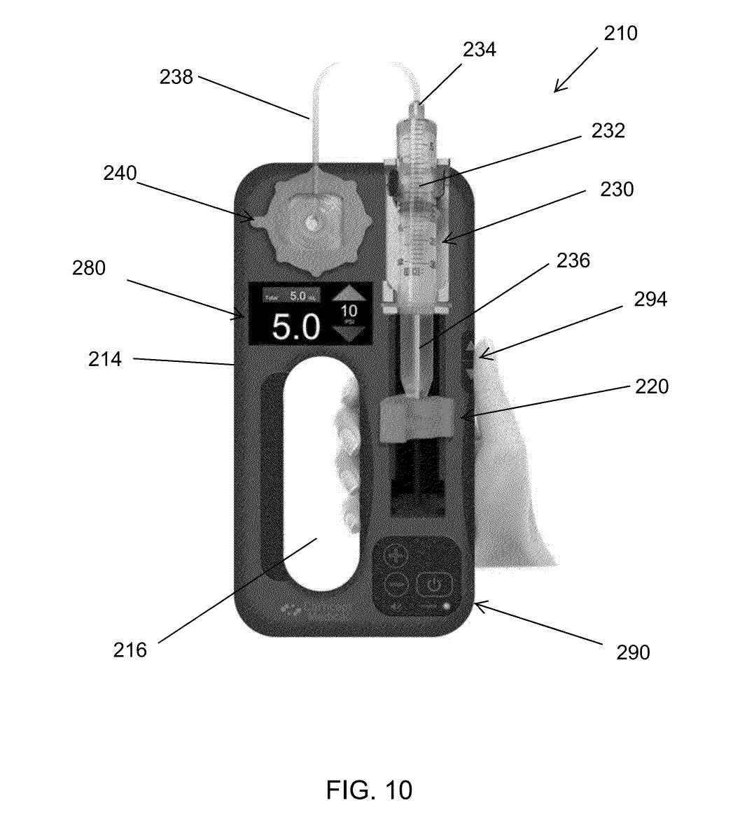

FIG. 10 illustrates a top view of another embodiment of an injection system 210 that is configured to be portable. For example, as shown, the housing 214 of the system 210 can be designed and otherwise configured to be grasped by a single hand of a user. Accordingly, in some embodiments, the system 210 can include one or more openings 216 and/or other ergonomic features that assist a user in grasping, manipulating and/or otherwise handling the system. As with the injection system described above with reference to FIG. 2, the housing 214 and/or another portion of the system 210 can be configured to securely receive one or more containers 230 containing anesthetics (e.g., nerve block agents) and/or any other fluids or materials. For example, as illustrated in FIG. 10, an upper surface of the fluid delivery module 210 can be designed and/or otherwise configured to receive a syringe 230. In some embodiments, the syringe 230 comprises a pre-filled syringe, as provided to the user by a drug manufacturer or supplier. Alternatively, however, such syringes or other fluid (e.g., nerve block or other anesthetic) containers can be filled on site by the user of the system. The syringes or other containers 230 configured to secure to the fluid delivery module 210 can be standard or non-standard, as desired or required for a particular application or use.

With continued reference to FIG. 10, the fluid delivery module 210 can comprise a housing or other outer portion 214 that encloses one or more internal components, such as, for example, motors, actuators, processors, memory units, controllers, wires or other electrical connections and components and/or the like. In embodiments, a plunger or other movable member 236 of the container 230 (e.g., syringe) can include one or more features or portions that are sized, shaped and/or otherwise adapted to engage an actuator assembly 220 of the motor (not shown). As shown, the actuator assembly 220 can include an actuator that is sized, shaped, positioned and/or otherwise configured to engage a plunger or other portion of the syringe 230 or other container loaded on a corresponding receiving area of the injection system. For example, in the depicted embodiment, a proximal end of the plunger is designed to be placed within a corresponding recess or opening of an actuator assembly 120. As discussed with reference to other arrangements herein, the actuator can be integrated with and/or otherwise coupled (e.g., directly or indirectly) to one or more motors of the module 210. In some embodiments, the motor of the fluid delivery module 210 comprises a stepper motor or another type of syringe motor that is capable of delivering and/or aspirating fluids against the expected forces during an injection/aspiration procedure. In some embodiments, as shown in FIG. 5, a locking device can be used to provide additional security that the plunger and/or other movable member of the container remains properly positioned relative to the actuator during use.

According to some embodiments, as discussed herein with reference to FIGS. 3 to 7, a pressure detection assembly 240 can be positioned between the syringe or other fluid container 230 and the conduit (e.g., tubing, handpiece, etc.—not shown) that transfers fluid from and/or to the subject. As shown, the pressure detection assembly 240 can be positioned downstream of the fluid container 230 (e.g., syringe). The pressure detection assembly 240 can be directly or indirectly coupled to the syringe or other container 230, as desired or required. For example, unlike the embodiment of FIG. 2, which illustrates the pressure detection assembly directly and physically coupled to the distal end (e.g., luer lock or other coupling) of the syringe, the pressure detection assembly 240 of FIG. 10 is indirectly coupled to the syringe or other container 230. In the illustrated arrangement, for instance, a section of tubing or other fluid conduit places the syringe or other fluid container in fluid communication with the pressure detection assembly 230. The syringe and the pressure detection assembly can be directly or indirectly coupled to each other for any of the embodiments disclosed herein or equivalents thereof, as desired or required by a particular design or configuration.

In some embodiments, as illustrated in FIGS. 3 to 7, the container (e.g., syringe) is integrated, at least in part, into a single housing or structure with the pressure detection assembly. However, in other embodiments, the pressure detection assembly can be positioned at a different location relative to the container 230 and/or the fluid delivery module 210. For instance, the pressure detection assembly 240 can be located along the fluid conduit (not shown) that places the syringe or other container 230 secured to the fluid delivery module 210 in fluid communication with a conduit and needle (not shown).

As discussed herein, the pressure detection assembly 240 can be configured to accurately detect a fluid pressure (e.g., positive or negative) being transferred by the fluid delivery module system 210. In some embodiments, a portion of the pressure detection assembly 240 is configured to come in contact (e.g., directly or indirectly) with the fluid being transferred through or near the pressure detection assembly. Such components or portions of the assembly 240 can be configured to be disposable or replaceable between procedures. Further, in some embodiments, the pressure detection assembly 240 comprises one or more reusable components or portions, such as, for example, a pressure sensor (not shown), associated electric connections, electronic components and/or the like. In such configurations, the reusable components or portions (e.g., sensor) can be integrated directly into the fluid delivery module 210 or any other portion or component of the injection system. Additional details regarding the configuration and operation of at least some embodiments of a pressure detection assembly 240 that can be incorporated into the injection/aspiration system are provided with reference to FIGS. 6 and 7 herein.

As shown in FIG. 10, the injection system 210 can include one or more displays or other output components or devices 280. For example, such an output device 280 can include a touchscreen display and/or any other type of display or screen. In some embodiments, one or more output devices 280 can also be configured to receive input from a user. For instance, as noted above, an output 280 can comprise a touchscreen that is configured to allow a user to make selections (e.g., via softkeys, virtual buttons of the touchscreen and/or the like).

The system 210 can include one or more input devices, components and/or features. For example, as noted above, the touchscreen or other output 280 can also be configured to receive instructions from a user. In addition, as shown in FIG. 10, the system 210 can include one or more additional buttons and/or other input devices or components. For example, the illustrated embodiment includes a button assembly 294 along one end of the housing 214. The button assembly 294 can include one or more buttons, dials, switches and/or the like to permit a user to make selections and/or otherwise control the operation of the system 210, as desired or required. In the illustrated arrangement, the button assembly 294 comprises up and down buttons, which can be used by the practitioner to, among other things, activate or deactivate an injection or aspiration procedure, increase or decrease an operational parameter and/or make any other adjustments permitted by the system.

As shown, the system 210 can also include additional buttons and/or other controllers 290, as desired or required. For example, the housing 214 can include a different region having one or more button, switches, dials and/or the like. In the illustrated arrangement, the system comprises a control region 290 comprises an on/off button, selector buttons (e.g., up and down buttons) and/or the like. Additional or fewer (and/or different) buttons and/or other control features can be incorporated into a particular system design or configuration.

As with any other embodiments disclosed herein, the system 210 of FIG. 10 can include one or more disposable or removably portions. For example, any components or portions of the system 210 that are configured to come in contact with fluids being transferred to or from a subject can be configured to be replaced after a procedure has bene completed. For example, in some embodiments, the syringe or other fluid container 230, the pressure detection assembly 240, the fluid conduit 234 placing the container in fluid communication with the pressure detection assembly and/or any other component of the system (e.g., downstream tubing or other handpiece assembly that is configured to be secured to the output of the pressure detection assembly—not shown) can be removable and disposable. Thus, the remaining portion of the system can be reused. In some embodiments, the removable (e.g., disposable) components and/or portions of the system can be quickly and easily removed (and replaced with new, sterile components and/or portions) to facilitate the execution of subsequent injection/aspiration procedures.

FIG. 11 illustrates another embodiment of a portable or handheld injection/aspiration system 310 similar to the one described herein with reference to FIG. 10. As shown, the system 310 can include a housing 314 that comprises a loading area along its upper surface for receiving a syringe or other fluid container 330. The various injection/aspiration systems disclosed herein can comprise loading areas that are configured to receive syringes or other containers (e.g., standard or non-standard) of varying sizes, shape and/or configurations. In some embodiments, in order to provide the system with the necessary information to perform a desired injection or aspiration procedure, a user is prompted to provide certain information regarding the particular syringe or other container that has been secured to the corresponding loading area of the system. For example, a user output/input device or component (e.g., a touchscreen) can include various standard or non-standard (e.g., as included by the particular user) types of syringes or other containers from which a user can choose.

With continued reference to FIG. 11, as with other embodiments discussed herein, the system 310 can include a pressure detection assembly 340, one or more input and/or output devices or components 380, 394 (e.g., touchscreen(s), button(s), other controller(s) and/or the like) that permit data and/or other information to be provide to and/or from the user. In some embodiments, as shown in FIG. 11, the pressure detection assembly 340 can include a fluid connector or other coupling 341 (e.g., a luer lock, another standard or non-standard connector, etc.) that permits easy and quick connection to a downstream handpiece or other fluid connector, as desired or required.

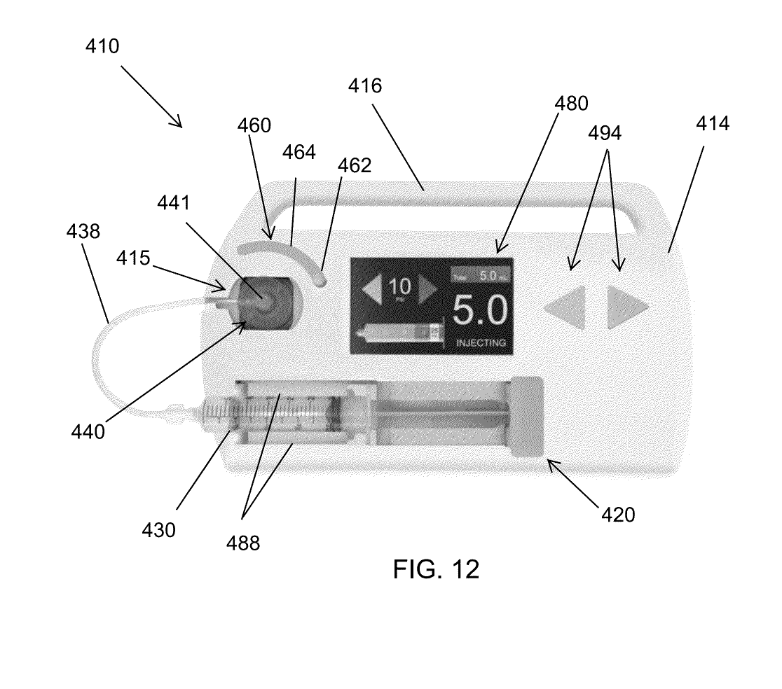

FIG. 12 illustrates yet another embodiment of a portable or handheld injection/aspiration system 410 similar to the ones described herein with reference to FIGS. 10 and 11. As shown, the system 410 can include a housing 414 that comprises a loading area along its upper surface for receiving a syringe or other fluid container 430. As discussed, this and/or other injection/aspiration systems disclosed herein can comprise loading areas that are configured to receive syringes or other containers (e.g., standard or non-standard) of varying sizes, shape and/or configurations. In some embodiments, in order to provide the system with the necessary information to perform a desired injection or aspiration procedure, a user is prompted to provide certain information regarding the particular syringe or other container that has been secured to the corresponding loading area of the system. For example, a user output/input device or component (e.g., a touchscreen) 480 can include various standard or non-standard (e.g., as included by the particular user) types of syringes or other containers from which a user can choose.

As with other embodiments discussed herein, the depicted system 410 can include a pressure detection assembly 440, one or more input and/or output devices or components 480, 494 (e.g., touchscreen(s), button(s), other controller(s) and/or the like) that permit data and/or other information to be provide to and/or from the user. In some embodiments, as shown in FIG. 12, the pressure detection assembly 440 can include a fluid connector or other coupling 441 (e.g., a luer lock, another standard or non-standard connector, etc.) that permits easy and quick connection to a downstream handpiece or other fluid connector, as desired or required.

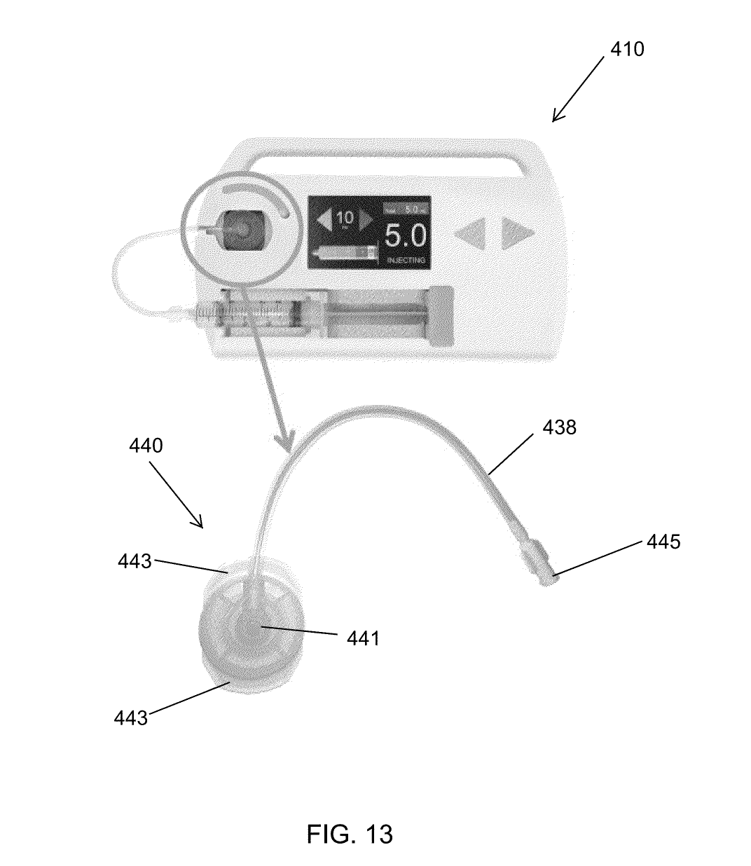

With continued reference to FIGS. 12 and 13, in some embodiments, the pressure detection assembly 440 can be configured to be a disposable component that can be secured to and removed from a corresponding recess or other receiving area 415 of the housing 414. As shown, a base portion of the pressure detection assembly 440 can comprise one or more tabs, flanges, wings and/or other engagement members 443 that help properly align, orient and/or position the assembly 440 within the recess or other receiving area 415 of the system 410. In some embodiments, any of the systems disclosed herein or equivalents thereof can comprise a locking assembly 460 can helps secure (e.g., temporarily lock) the pressure detection assembly 440 within or relative to the housing 414. The locking assembly 460 can include a movable member 462 that is configured to slide within a corresponding groove or slot 464 (e.g., between “locked” and “unlocked” positions or orientations). Thus, in some arrangements, once a user inserts a new (e.g., sterile) pressure detection assembly 440 within the receiving area 415 of the housing 414, the user can slide or otherwise move the movable member 462 of the locking assembly 460 to a locked position to ensure that the pressure detection assembly 440 is properly seated, positioned, aligned and/or otherwise oriented prior to use. In some embodiments, the locking assembly 460 (e.g., either alone or in conjunction with the pressure detection assembly 440 positioned within the receiving area 415) can include a cammed or other locking or engagement portion or feature to inform (e.g., tactically, audibly, etc.) the user that the assembly 460 has been properly secured within the system 410.

In some configurations, once an injection/aspiration procedure has been completed, the user can move the locking assembly 460 into an unlocked position in order to release and remove the pressure detection assembly 440 from the housing 414. A new disposable pressure detection assembly 440 can be inserted within the housing in preparation for a subsequent procedure. Such features relating to a disposable pressure detection assembly that is configured to be positioned (and/or locked) within a housing of an injection/aspiration system (e.g., with or without a locking assembly) can be incorporated into any of the systems disclosed herein or equivalents thereof.

As illustrated in FIGS. 11 and 12, the pressure detection assembly 340, 440 can include a pigtail or extension portion 338, 438 that connects the main portion of the assembly 340, 440 to the distal end of the fluid container 330, 430 secured to the system. Such a configuration can permit the system 310, 410 to be advantageously reduced in size while still allowing for a relatively large container 330, 430 to be incorporated into the system. For example, by using a pigtail or extension 338, 438, the need to have an in-line orientation between the distal end of the fluid container (e.g., syringe) 330, 430 and the pressure detection assembly 340, 440 can be eliminated. This can permit the length (and/or other dimensions) of the housing, and thus the overall injection/aspiration system) to be advantageously reduced. Such a design can be particularly helpful for portable systems, where size, weight and/or otherwise a smaller footprint is desired or required.

With continued reference to FIG. 13, the pigtail or extension 438 can be integrated and provided together with (e.g., as a single package) the pressure detection assembly 440. Such a design can be incorporated in any of the system embodiments disclosed herein or equivalents thereof. In other embodiments, however, such a pigtail or extension can be integrated with the syringe or other fluid container 430 or can be separate from both the container 430 and the pressure detection assembly 440, as desired or required. As also illustrated in FIG. 13, any pressure detection assembly 440 disclosed herein can comprise a luer or other standard or non-standard coupling or other connector 441. Such a coupling or connector 441 can be sized, shaped, orientated (e.g., vertically, horizontally, diagonally, etc.) and/or otherwise configured to permit a fluid conduit to be secure thereto.

As discussed herein, any injection/aspiration systems can be designed and otherwise adapted to be portable and relatively compact. Such configurations can permit the units to be easily transported to facilitate use between different facilities or locations within a single facility. Thus, any of the systems disclosed herein or equivalents thereof can be relatively small in size (e.g., with the assistance of features such as the pigtail or extension to reduce length), can be relatively light (e.g., about or less than 5 pounds), can include handles or other graspable features 316, 416 (see FIGS. 11 and 12) to facilitate safe and convenient handling, carrying and transport, can include internal batteries (not shown) such as rechargeable lithium or other batteries and/or can include one or more other features, components or designs, as desired or required.

Any of the system embodiments disclosed herein can also include additional visual confirmation regarding one or more aspects of a procedure being conducted. Such visual confirmation can be in addition to or in lieu of additional features or components (e.g., information provided via touchscreen 480 or other screens or outputs that are incorporated within the system or separate from the system, voice-enabled alerts, alarms and/or other audible alerts, haptic or other tactile alerts, etc.). For example, as illustrated in FIG. 12, one or more portions of the housing can be configured to emit a light that is indicative of one or more system conditions. In the depicted embodiments, such portions 488 are at or near the loading area where the syringe or other fluid container 430 is secured. However, in other arrangements, portions capable of being lighted or otherwise configured to emit a visual signal can be positioned along any other portion of the system or related components (e.g., around a periphery of the housing, along the handle, around the touchscreen or other visual output, around, at or near the buttons 384, foot pedal or other controller, a separate screen or other output device, etc.), either in lieu of or in addition to the region surrounding the fluid container 430, as desired or required. In FIG. 12, both the area or region 488 surrounding or near the fluid container 430 and the buttons 494 that permit a user to modify an operational parameter of the system are configured to change color (e.g., using LEDs and/or other lighting technology).

In some embodiments, for example, the region 488 surrounding the container 430, the buttons 494 and/or any other portion of the system that is configured to emit a visual signal can be configured to change colors (and/or intensity or hue of the same color, in some arrangements) depending on the status of an injection/aspiration procedure. For example, in some embodiments, such “backlight” or other visual configuration can be configured to emit a first color (e.g., green) when fluid is being delivered by the system to a subject, a second color (e.g., blue) when fluid is being aspirated by the system from a subject, and a third color (e.g., red) when the threshold positive or negative pressure (e.g., maximum pressure or vacuum) is achieved. Thus, a user can be always informed regarding the status of a procedure. This can allow for procedures to be conducted in an easier, safer and better informed manner, allowing the user to focus on the actual injection and/or aspiration without having to continuously look at the touchscreen or other output. As noted, light emitting diodes (LEDs) and/or any other source of light can be used to create the backlight or other visual effect. In addition, more or fewer than 3 (e.g., 2, 3, 4, 5, 6, more than 6) light colors (and/or hues, intensities, etc.) can be used in a particular system, as desired or required.

With any of the portable injection/aspiration system configurations shown or contemplated herein (e.g., the systems 210, 310, 410 illustrated and described herein with reference to FIGS. 10, 11 and 12), power can be supplied to the various components via one or more batteries. For example, in some embodiments, such batteries can be permanently or removably positioned within and/or near the housing of the respective system. In some embodiments, such batteries or other power sources can be rechargeable, irrespective of whether they are configured for convenient removal from the system. In other embodiments, however, the various systems disclosed herein, including the portable systems are configured to have supplied to them via a hardwired connection (e.g., to an AC plug or other outlet, a separate power supply, etc.), as desired or required.

In some embodiments, the use of portable systems (e.g., systems that can be easily grasped and manipulated) can provide certain advantages and/or other benefits to a user and/or a subject. For example, such configuration can permit a surgeon, other physician or practitioner and/or other user to easily transport an injection/aspiration system outside a facility (e.g., clinic, hospital, doctor's office, etc.) where such procedures are traditionally performed. Alternatively, such configurations can permit practitioners to conveniently use the system within a particular facility (e.g., as the system can be easily moved between examination or hospital rooms) of a particular hospital, clinic and/or other facility.

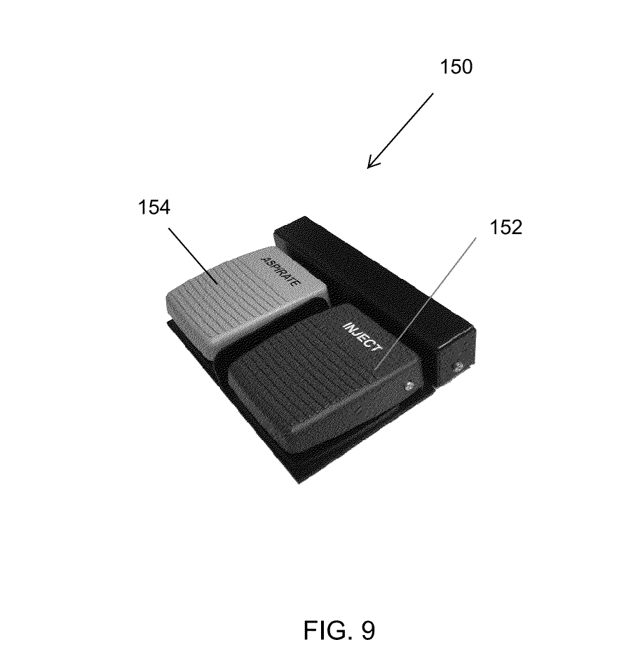

FIG. 9 illustrates one embodiment of a foot pedal assembly 150 having two separate pedals or controllers 152, 154 that can be manipulated by a user to perform an injection procedure. For example, in the illustrated embodiment, the controllers include foot-actuated pedal that can be depressed by a user to either inject or aspirate. However, as noted herein, one or more other types of controllers can be used to allow a user to execute a procedure, such as for example, hand-operated controllers (e.g., buttons, dials, touchscreens, etc.), voice-activated controller and/or the like. Regardless of their exact type and/or configuration, the controllers 150 can be operatively coupled to a processor or control unit of the injection system 100.

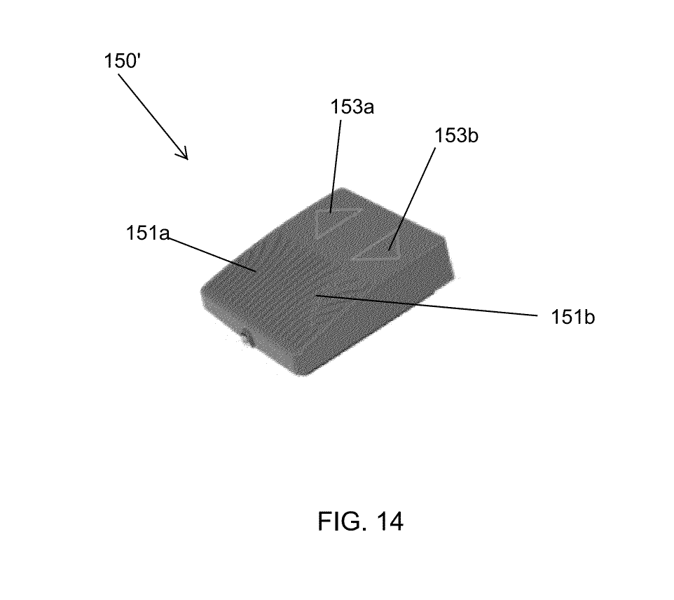

According to some configurations, as illustrated in FIG. 14, any of the injection system embodiments disclosed herein can include a foot pedal 150′ comprising a single pedal design. For example, a foot pedal 150′ can include a single maneuverable or otherwise movable foot pedal 151. In some embodiments, such a single foot pedal 150′ can be configured to be moved in two or more (e.g., 3, 4, 5, etc.) directions (e.g., left, right, up, down, etc.), as desired or required. Thus, for any of the embodiments disclosed herein, a single foot pedal 150′ can be used to regulate two or more aspects of the operation of the system. For example, as illustrated in FIG. 14, a single foot pedal 150′ can include a rocking configuration or design, such that it can be depressed (e.g., downwardly) either on its left side 153a or its right side 153b. In the depicted embodiment, by stepping (e.g., pressing) the foot pedal 150′ downwardly on the left side 153a of the pedal, the user can aspirate fluid from the subject using the system. For instance, as shown, the pedal 150′ can include one or more indicators 155a, 155b that are representative of a particular function or other controllable aspect of the system, such as, an aspiration symbol or indicator 155a and a delivery symbol or indicator 155b. In other embodiments, the foot pedal 150′ is configured to be pressed or otherwise moved in a different direction than left and right, such as, for example, top and bottom (e.g., when viewed from the top), up and down (e.g., when viewed from the side) and/or the like, as desired or required.

The level of control over one or more aspects of the injection/aspiration system provided by a pedal 150′ can vary. For example, the single foot pedal configuration, such as the one illustrated in FIG. 14, can be designed to simply permit a user to select between fluid delivery (if, e.g., the right portion or side 153b of the pedal 150′ is pressed) and fluid aspiration (if, e.g., the left portion or side 153a is pressed). In some embodiments, the pedal can be further configured to permit a user to select one or more additional functions related to control of the injection system (e.g., the rate of fluid delivery or aspiration based on how far the pedal is depressed or otherwise actuated), as desired or required.

According to some embodiments, for example, a processor of the system 100 is operated according to an algorithm or other operational scheme that seeks to prevent damage to the disposables and hardware by keeping the operating pressure of the fluid (e.g., both positive pressure during fluid delivery and negative pressure during aspiration) within a safe value or range. In some embodiments, the fluid pressure during delivery of nerve block agents, other anesthetic or agents and/or other fluids is generally constant. Thus, in such configurations, the speed at which the actuator, and thus the plunger of the syringe or other fluid container, is moved can vary in order to maintain a constant or generally constant fluid pressure during an injection procedure. If resistance is encountered, the linear actuator speed will automatically slow or otherwise adjust. For example, if an occlusion or other incident that causes pressure to increase above a particular threshold, the actuator can be stopped to prevent damage to the subject and/or the injection system. According to some embodiments, the system is configured to maintain a positive fluid pressure within the fluid container and/or the adjacent fluid conduit (e.g., tubing) that transfers fluid from the container to the needle at a pressure of about 0 to 20 psi (e.g., 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20 psi, pressures between the foregoing, etc.). As discussed, a processor or other control unit of the fluid delivery module or other component of the injection system can be configured to maintain a generally constant pressure during fluid delivery or aspiration. In other arrangements, the system is configured to at least prevent the delivery (e.g., positive) pressure and/or the aspiration (e.g., negative) pressure from going above or below a particular threshold (e.g., maximum or minimum level or range). In other embodiments, however, the system is configured to maintain the pressure at value greater than 20 psi (e.g., 20-25, 25-30, 30-40, 40-50, 50-75, 75-100 psi, pressures between the foregoing, greater than 100 psi, etc.) during fluid delivery, as desired or required.

In some embodiments, during aspiration, the system is configured to maintain a negative fluid pressure within the fluid container and/or the adjacent fluid conduit (e.g., tubing) at a pressure of about 0 to −5 psi (e.g., 0 to −1, −1 to −2, −2 to −3, −3 to −4, −4 to −5 psi, pressures between the foregoing, etc.). As discussed, a processor or other control unit of the fluid delivery module or other component of the injection system can be configured to maintain a generally constant pressure during fluid delivery or aspiration. In other arrangements, the system is configured to at least prevent the delivery (e.g., positive) pressure and/or the aspiration (e.g., negative) pressure from going above or below a particular threshold (e.g., maximum or minimum level or range). In other embodiments, however, the system is configured to maintain the pressure at value greater than −5 psi (e.g., −5 to −10, −10 to −20, −20 to −30, −30 to −40, −40 to −50 psi, pressures between the foregoing, less than −50 psi, etc.) during aspiration, as desired or required.

According to some embodiments, the system is configured to maintain the maximum or threshold positive pressure during fluid delivery to 20 to 40 psi (e.g., 20-30, 20-25, 25-30, 30-40 psi, pressures between the foregoing, etc.). In some embodiments, the system is configured to maintain the maximum or threshold positive pressure during fluid delivery above 40 psi (e.g., 40-50, 50-75, 75-100 psi, pressures between the foregoing, pressures greater than 100 psi, etc.). Further, according to some embodiments, the system is configured to maintain the minimum or threshold negative pressure during aspiration to −5 to −10 psi (e.g., −5 to −6, −6 to −7, −7 to −8, −8 to −9, −9 to −10, −5 to −7 psi, pressures between the foregoing, etc.). In some embodiments, the system is configured to maintain the minimum or threshold positive pressure during fluid delivery below −10 psi (e.g., −10 to −20, −20 to −30, −30 to −40, −40 to −50, −50 to −75, −75 to −100 psi, pressures between the foregoing, pressures less than 100 psi, etc.).

According to some embodiments, the processor is configured to operate the fluid delivery module (e.g., the motor(s), actuator(s) and/or other components contained therein) to eliminate or reduce residual fluid drip from the needle at the completion of delivery. For example, in some embodiments, when a stop command is issued, the processor can regulate the speed of the actuator/motor to achieve a pressure that prevents dripping (e.g., 0 to 1 psi).

In some embodiments, as discussed herein, aspiration, or application of a slight negative pressure, is used in the nerve block procedures to confirm needle placement, specifically whether the needle is positioned in a blood vessel. The feature allows the device to quickly create and to stay at a slight negative pressure. The pressure can be regulated to be equivalent to the negative pressure that a nurse uses during an actual nerve block procedure.

In some embodiments, the processor is configured to operate the injection system based on a maximum or threshold fluid pressure. Such a threshold pressure value can be pre-programmed into a system. However, in alternative embodiments, a user can adjust or otherwise select such a maximum or threshold fluid pressure, based on his or her specific requirements, desires or preferences. According to some embodiments, the system is configured to maintain the maximum or threshold positive pressure during fluid delivery to 20 to 40 psi (e.g., 20-30, 20-25, 25-30, 30-40 psi, pressures between the foregoing, etc.). In some embodiments, the system is configured to maintain the maximum or threshold positive pressure during fluid delivery above 40 psi (e.g., 40-50, 50-75, 75-100 psi, pressures between the foregoing, pressures greater than 100 psi, etc.). Further, according to some embodiments, the system is configured to maintain the minimum or threshold negative pressure during aspiration to −5 to −10 psi (e.g., −5 to −6, −6 to −7, −7 to −8, −8 to −9, −9 to −10, −5 to −7 psi, pressures between the foregoing, etc.). In some embodiments, the system is configured to maintain the minimum or threshold positive pressure during fluid delivery below −10 psi (e.g., −10 to −20, −20 to −30, −30 to −40, −40 to −50, −50 to −75, −75 to −100 psi, pressures between the foregoing, pressures less than 100 psi, etc.).

According to some embodiments, as noted above, during the delivery of fluid (e.g., nerve block agents, other anesthetic, etc.) into a subject and/or aspiration of fluid from a subject (e.g., to confirm that the needle in fluid communication with the fluid delivery module is not located in a blood vessel or other undesirable or dangerous location of the subject's anatomy), the processor is configured to operate the actuator and motor of the fluid delivery module 110 at a constant or generally constant pressure (e.g., positive or negative pressure). This can help create a safe and predictable approach that emulates or otherwise simulates how a procedure would be executed manually by a physician, nurse or other practitioner). In some embodiments, the processor is configured to maintain such positive and/or negative pressure by allowing the actuator and/or motor of the fluid delivery module to slip or otherwise slow down or stop when a pressure exceeds (or dips below) a threshold level.

According to any of the embodiments disclosed herein, the injection system is configured to accurately detect pressure of the fluid being transferred to and/or from the subject by having a pressure detection assembly that at least partially contacts the fluid. In some embodiments, the injection system comprises a pressure detection assembly that can be at least partially reused (e.g., is at least partially disposable and reusable). In some embodiments, the injection system is configured to deliver and/or aspirate fluids to and/or from a subject by not maintaining a constant delivery rate (e.g., volumetric delivery rate) and/or speed. In some embodiments, fluid is transferred to and/or from the subject using an injection system that maintains a generally constant pressure (e.g., positive or negative pressure, depending on whether fluids are being delivered or aspirated).

In any of the embodiments disclosed herein, an injection system comprises a nerve stimulator or similar electrical stimulation device. Such a stimulator or other stimulation device can be electrically coupled to a stimulation needle which can be used for delivering anesthetics (and/or other fluids) and for selective nerve stimulation of the subject's anatomy. During the administration of nerve block agents, other anesthetics, electrical stimulation can be used to verify the proximity of the needle to the target nerve, advantageously increasing the success rate of a nerve block procedure. For example, since stimulation of nerves elicits different muscle responses, twitches and/or other reactions by the patient, an electrical impulse delivered to or near the tip of the needle can allow the anesthesiologist or other clinical performing the procedure to determine how close the needle is relative to the target nerve tissue. Accordingly, targeted or nearby nerve tissue can be identified by observing the patient for the desired corresponding muscle response.

In such embodiments that comprise a stimulation needle, the type, size, length, and/or other details of the needle can be selected to allow for improved nerve stimulation and delivery of anesthetic agents. For example, the needle can be insulated over a majority of its length and have a conductive area at the tip to localize stimulation to the needle tip where the anesthetic will also be delivered. In some embodiments, a stimulation needle can include a needle body, a handpiece or needle hub, tubing or other conduit, and a lead. The tubing or other conduit can be configured to place the handpiece or needle hub and needle body in fluid communication with the anesthetic source (e.g., anesthetic agents and/or other fluids loaded onto a corresponding fluid delivery module, via one or more fluid conduits, handpiece and/or the like, etc.). Further, the one or more electrical leads can place the handpiece, needle hub, needle body (e.g., when no handpiece or needle hub is used) and/or the like in electrical communication with the stimulator. As noted, in some embodiments, the stimulation needle and/or any other needle used in an injection system disclosed herein can be used with or without a handpiece, hub or other intermediate component. Thus, such needles can be directly or indirectly coupled to a fluid conduit (e.g., tubing) and/or other fluid passage that is in fluid communication with an anesthetic or other fluid source.

According to some embodiments, an anesthetic or other type of injection system comprises a stand-alone nerve stimulator operatively coupled to, for example, a fluid delivery module. The stimulation needle tubing can be coupled to and placed in fluid communication with the delivery line or conduit in fluid communication with the fluid delivery module, for example, via luer fittings, other suitable fittings, etc. The one or more stimulation needle leads can be coupled to an output of the stimulator. In some embodiments, the nerve stimulator and fluid delivery module are operatively coupled and communicate with one another via a hardwired connection (e.g., USB, ethernet, etc.) or a wireless connection (e.g., Bluetooth, radio frequency, etc.).

In some embodiments, a stimulator can be at least partially integrated into a fluid delivery module to form a single, integrated or unitary unit. In some such embodiments, the one or more fluid conduits and/or stimulator leads can be coupled (e.g., removably or permanently) to one another (e.g., along at least a part of their lengths or other portion). Further, along their distal end, such fluid conduits and/or electrical leads and can be coupled to a proximal end or other portion of a handpiece or other conduit that is in fluid communication with a fluid delivery module of the nerve block injection system. Alternatively, such delivery lines or conduits and/or stimulator leads can be independent of one another. In some embodiments, the stimulation needle body can be coupled to a distal end of the handpiece. The handpiece can be configured to place the needle body in fluid communication with the delivery line and in electrical communication with the stimulator lead. In some embodiments, stimulation information, for example, one or more stimulator parameters (e.g., the strength of the electrical impulse being provided to the needle, historical information regarding the level of electrical impulse delivered to the needle, etc.) can be displayed on a display (e.g., incorporated into the injection system or operatively coupled to the injection system) and/or any other output (e.g., other display, printout, etc.). In some embodiments, information regarding the amount of electrical stimulation delivered to the patient can be stored and saved on a memory associated with the system (e.g., internal memory, external memory, etc.) and/or delivered to another location (e.g., over a network).

A nerve stimulator can be configured to provide the needle with electrical current having an impulse amplitude in the range of about 0 mA to about 10 mA (e.g., about 0, 0.1, 0.2, 0.3, 0.4, 0.5, 1, 2, 3, 4, 5, 6, 7, 8, 9, 10 mA, values between the foregoing, etc.). In some embodiments, a relatively low current range, such as, for example, 0-5 mA, can help reduce the risk of trauma to the nerves. In some embodiments, use of a relatively higher current intensity (for example, greater than 1.0 mA in some applications) can result in an exaggerated muscle response or twitch and increased discomfort for the patient. However, impulse amplitudes greater than about 5 or 10 mA can be used in the conjunction with the various injection system embodiments disclosed herein, as desired or required. In some embodiments, the stimulator is configured to compensate for variations in tissue resistance within the patient's anatomy to supply current having a substantially or generally constant amplitude. In some embodiments, the nerve stimulator is configured to deliver stimulus pulses having a duration of about 0.1 ms. A short stimulus pulse duration can advantageously help cause motor nerve response rather than sensory nerve response. Other pulse durations are also possible. In some embodiments, the stimulator is configured to deliver impulses to the needle at a frequency of 1 Hz or 2 Hz. In some embodiments, the stimulator allows for the selection of a 1 Hz or 2 Hz pulse frequency, for example, via a switch, button, or other controller. In some embodiments, other pulse frequencies are also possible.

For any of the embodiments of the nerve block or other fluid injection systems disclosed herein, details related to a specific injection procedure can be recorded, maintained and/or otherwise memorialized. For example, the injection system can be configured to receive and maintain the name of the patient, the date and time that the procedure was performed, the duration of the procedure, the physicians, clinicians and/or other personnel that participated in the preparation and/or execution of the procedure, the disease or condition being treated, specific treatment codes and other administrative information and/or the like. Such data collection capabilities can assist with billing, insurance processing, patient record keeping, generation of reports, reordering of medicaments and other injectable materials and/or other functions. In some embodiments, such records or summaries (e.g., printouts, electronic file, etc.) can be included in or otherwise connected with (e.g., physically, electronically, etc.) a patient's file or chart. In addition, the use of the summaries or reports can provide one or more additional benefits to a user. For example, such summaries and reports can improve the economic return on an injection procedure for the service provider by leveraging the relatively favorable reimbursement of the corresponding ultrasound-guided (or other imaging-guided) procedures.

According to some embodiments, an injection system includes a printer, another output device, memory and/or the like to help memorialize the details associated with a specific injection procedure. As noted herein, the corresponding output resulting from such recordkeeping can assist with billing, insurance processing, patient record keeping, generation of reports and/or the like. In addition, such printouts or alternative forms of output (e.g., electronic reports) can memorialize the details of a particular procedure, serving as evidence of what was performed (e.g., which and how much of each nerve block agent, other anesthetic or other medicament and/or other substance was injected, visual confirmation via an ultrasound or other imaging technology of the needle location and other details of the injection, etc.), to whom the injection was administered, who performed the injection procedure, when and where the procedure was executed and/or the like. As noted above, such summaries can be provided on a paper printout (e.g., a printer that is incorporated with or operatively coupled to an injection system), electronic form (e.g., a summary generated as a pdf, an image or some other standard or non-standard viewable format, etc.) and/or the like.

In any of the embodiments disclosed herein, one or more components, including a processor, computer-readable medium or other memory, controllers (for example, dials, switches, knobs, etc.), displays and/or the like are incorporated into and/or coupled with (for example, reversibly or irreversibly) one or more modules and/or components of the system.

In some embodiments, the system comprises various features that are present as single features (as opposed to multiple features). For example, in one embodiment, the system includes a single housing configured to receive a single pressure detection assembly (in some arrangements, the pressure detection assembly comprises a single membrane), a single fluid connection between a single syringe or other fluid container (e.g., prefilled syringe) and the pressure detection assembly, a single outlet from the pressure detection assembly configured to connect to a fluid conduit (e.g., with or without a handpiece), a single pressure sensor (e.g., that is configured to detect in real-time, and without need for modeling or estimation the positive or negative pressure within the fluid being transferred), a single controller or set of controllers (e.g., buttons, touchscreen, etc.) that permit a user to regulate one or more aspects of the system and/or the like.

Although several embodiments and examples are disclosed herein, the present application extends beyond the specifically disclosed embodiments to other alternative embodiments and/or uses of the various inventions and modifications, and/or equivalents thereof. It is also contemplated that various combinations or subcombinations of the specific features and aspects of the embodiments may be made and still fall within the scope of the inventions. Accordingly, various features and aspects of the disclosed embodiments can be combined with or substituted for one another in order to form varying modes of the disclosed inventions. Thus, the scope of the various inventions disclosed herein should not be limited by any particular embodiments described above. While the embodiments disclosed herein are susceptible to various modifications, and alternative forms, specific examples thereof have been shown in the drawings and are described in detail herein. However, the inventions of the present application are not limited to the particular forms or methods disclosed, but, to the contrary, cover all modifications, equivalents, and alternatives falling within the spirit and scope of the various embodiments described and the appended claims. Further, the disclosure herein of any particular feature, aspect, method, property, characteristic, quality, attribute, element and/or the like in connection with an implementation or embodiment can be used in all other implementations or embodiments set forth herein.

In any methods disclosed herein, the acts or operations can be performed in any suitable sequence and are not necessarily limited to any particular disclosed sequence and not be performed in the order recited. Various operations can be described as multiple discrete operations in turn, in a manner that can be helpful in understanding certain embodiments; however, the order of description should not be construed to imply that these operations are order dependent. Additionally, any structures described herein can be embodied as integrated components or as separate components. For purposes of comparing various embodiments, certain aspects and advantages of these embodiments are described. Not necessarily all such aspects or advantages are achieved by any particular embodiment. Thus, for example, embodiments can be carried out in a manner that achieves or optimizes one advantage or group of advantages without necessarily achieving other advantages or groups of advantages.

The methods disclosed herein include certain actions taken by a practitioner; however, they can also include any third-party instruction of those actions, either expressly or by implication. For example, actions such as “advancing a needle within a subject,” “delivering a fluid to a subject” or “aspirating fluid from a subject” include “instructing advancing a needle within a subject,” “instructing delivering fluid to a subject” or “instructing aspirating fluid from a subject,” respectively. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (e.g., as accurate as reasonably possible under the circumstances, for example ±5%, ±10%, ±15%, etc.). For example, “about 1 mm” includes “1 mm.” Phrases preceded by a term such as “substantially” include the recited phrase and should be interpreted based on the circumstances (e.g., as much as reasonably possible under the circumstances). For example, “substantially rigid” includes “rigid,” and “substantially parallel” includes “parallel.”

What is claimed is:

1. An injection system configured to deliver at least one fluid to a subject, the system comprising:
a fluid delivery module configured to receive a fluid container, wherein once secured to the fluid delivery module, the fluid container is configured to engage an actuator of the fluid delivery module;
at least one motor coupled to the actuator, wherein the at least one motor is configured to selectively move the actuator to create a positive pressure within the fluid container;
wherein the fluid container is configured to be placed in fluid communication with a fluid conduit, a distal end of the fluid conduit being configured to receive a needle for placement within a target injection location of the subject;
a pressure detection assembly in fluid communication with the fluid container, wherein the pressure detection assembly is configured to detect the real-time pressure within the fluid container and the fluid conduit;
wherein the pressure detection assembly comprises a disposable portion and a reusable portion, the disposable portion comprising at least one membrane in fluid communication with fluid being transferred between the fluid container and the fluid conduit to permit for more accurate, real-time fluid pressure detection; and
wherein the reusable portion of the pressure detection assembly comprises a pressure sensor configured to be operatively coupled to the at least one membrane, the pressure sensor being configured to determine the real-time pressure within the fluid container and the fluid conduit; and
a processor configured to regulate at least one aspect of the injection system based on, at least in part, the real-time pressure detected by the pressure detection assembly.

2. The injection system of claim 1,
wherein the system is configured to aspirate a volume of fluid from a subject, wherein the pressure detection assembly is configured to detect a negative pressure within the fluid container and the fluid conduit;
wherein the processor is configured to maintain a generally constant positive pressure within the fluid container and the fluid conduit during delivery of a fluid to a subject;
wherein the processor is configured to maintain a variability of the positive pressure within the fluid container and the fluid conduit within a range of 1 to 4 psi; and
wherein the fluid container comprises a syringe, and wherein the actuator of the fluid delivery module is configured to engage a movable member of the syringe.

3. The injection system of claim 1, wherein the system is configured to aspirate a volume of fluid from a subject, wherein the pressure detection assembly is configured to detect a negative pressure within the fluid container and the fluid conduit.

4. The injection system of claim 1, wherein the processor is configured to maintain a generally constant positive pressure within the fluid container and the fluid conduit during delivery of a fluid to a subject.

5. The injection system of claim 4, wherein the processor is configured to maintain a variability of the positive pressure within the fluid container and the fluid conduit within a range of 1 to 4 psi.

6. The injection system of claim 1, wherein the fluid container comprises a syringe, and wherein the actuator of the fluid delivery module is configured to engage a movable member of the syringe.

7. The injection system of claim 1, wherein the fluid container comprises a standard or non-standard coupling configured to secure to a proximal end of the fluid conduit.

8. The injection system of claim 1, wherein the system comprises a visual indicator relating to a status of a procedure being performed using the system.

9. The injection system of claim 8, wherein the visual indicator comprises at least one light configured to change color depending on the status.

10. The injection system of claim 1, wherein the fluid container comprises a syringe, and wherein the actuator of the fluid delivery module is configured to engage a movable member of the syringe.

11. The injection system of claim 1, wherein the fluid container and at least a portion of the pressure detection assembly are included in a unitary assembly that is configured to secure to the fluid delivery module of the injection system.

12. The injection system of claim 1, wherein the at least one membrane of the pressure detection assembly is configured to be in fluid communication with a fluid being transferred between the container and the fluid conduit, the at least one membrane being configured to move upwardly or downwardly relative to the pressure sensor.

13. An injection system configured to deliver at least one fluid to a subject, the system comprising:
a fluid delivery module configured to receive a fluid container, wherein once secured to the fluid delivery module, the fluid container is configured to engage an actuator of the fluid delivery module;
at least one motor coupled to the actuator, wherein the at least one motor is configured to selectively move the actuator to create a positive pressure within the fluid container;
wherein the fluid container is configured to be placed in fluid communication with a fluid conduit, a distal end of the fluid conduit being configured to receive a needle for placement within a target injection location of the subject;
a pressure detection assembly in fluid communication with the fluid container, wherein the pressure detection assembly is configured to detect the real-time pressure within the fluid container and the fluid conduit; and
a processor configured to regulate at least one aspect of the injection system based on, at least in part, the real-time pressure detected by the pressure detection assembly;
wherein the injection system is configured to aspirate a volume of fluid from a subject, wherein the pressure detection assembly is configured to detect a negative pressure within the fluid container and the fluid conduit; and
wherein the processor is configured to maintain a generally constant positive pressure within the fluid container and the fluid conduit during delivery of a fluid to a subject to simulate a manually-executed injection procedure to further enhance the safety of an injection procedure.

14. The injection system of claim 13, wherein the pressure detection assembly comprises a disposable portion and a reusable portion, the disposable portion comprising at least one membrane in fluid communication with fluid being transferred between the fluid container and the fluid conduit.

15. The injection system of claim 13, wherein the processor is configured to maintain a variability of the positive pressure within the fluid container and the fluid conduit within a range of 1 to 4 psi.

16. An injection system of claim 13, wherein the fluid container comprises a syringe, and wherein the actuator of the fluid delivery module is configured to engage a movable member of the syringe.

17. A method of delivering at least one fluid to a subject, the system comprising:
delivering a fluid from a fluid delivery module of an injection system to a needle located along a distal end of a fluid conduit in fluid communication with the fluid delivery module, wherein the fluid delivery module is configured to receive a fluid container containing the fluid to be delivered to the subject, wherein once secured to the fluid delivery module, the fluid container is configured to engage an actuator of the fluid delivery module;
detecting the pressure in real-time of the fluid being transferred by the fluid delivery module to or from the subject via a pressure detection assembly in fluid communication with the fluid container, wherein the pressure detection assembly is configured to detect the real-time pressure within the fluid container and the fluid conduit; and
regulating at least one aspect of the injection system using a processor of the system based on, at least in part, the real-time pressure detected by the pressure detection assembly;
wherein the injection system is configured to aspirate a volume of fluid from a subject, wherein the pressure detection assembly is configured to detect a negative pressure within the fluid container and the fluid conduit; and
wherein the processor is configured to maintain a generally constant positive pressure within the fluid container and the fluid conduit during delivery of a fluid to a subject to simulate a manually-executed injection procedure to further enhance the safety of an injection procedure.

18. The method of claim 17, wherein the fluid delivery module comprises at least one motor coupled to the actuator, wherein the at least one motor is configured to selectively move the actuator to create a positive pressure within the fluid container.

19. The method of claim 17, further comprising aspirating fluid from the subject using the system prior to injecting fluid to the subject to ensure that the needle is properly and safely positioned within the subject.

20. The method of claims 17, wherein the pressure detection assembly comprises a disposable portion and a reusable portion, the disposable portion comprising at least one membrane in fluid communication with fluid being transferred between the fluid container and the fluid conduit.
